(19) United States
(12) Patent Application Publication
(10) Pub. No.: US 2026/0191972 A1
(43) Pub. Date: Jul. 9, 2026

(54) MULTI-ARM POLYETHYLENE GLYCOL-DRUG CONJUGATE

(71) Applicant: JENKEM TECHNOLOGY CO., LTD. (BEJING), Beijing (CN)

(72) Inventors: Xichun ZHENG, Beijing (CN); Meina LIN, Beijing (CN); Qingbin WANG, Beijing (CN); Jian XU, Beijing (CN); Hongli JIA, Beijing (CN); Jie WANG, Beijing (CN); Mai YANG, Beijing (CN); Biao LIU, Beijing (CN); Xuan ZHAO, Beijing (CN)

(21) Appl. No.: 19/131,860

(22) PCT Filed: Dec. 27, 2023
(86) PCT No.: PCT/CN2023/142478

§ 371 Date: May 21, 2025

(30) Foreign Application Priority Data
Dec. 30, 2022 (CN) 202211726183.6

(51) Int. Cl. A61K 47/60; A61K 47/68

(52) U.S. Cl. CPC A61K 47/60; A61K 47/6849

(57) ABSTRACT

Disclosed in the present invention are a multi-arm polyethylene glycol-drug conjugate, and a preparation method therefor and the use thereof. In the multi-arm polyethylene glycol-drug conjugate provided in the present invention, the terminal position of the multi-arm polyethylene glycol structure thereof may be a heterofunctional group, so that two or even three drugs can be simultaneously connected in one molecular system, thereby achieving the aim of treating multiple diseases with one drug. In addition, compared with a linear multi-arm polyethylene glycol-drug conjugate, the multi-arm PEG has a plurality of terminal groups, has a plurality of introduction points of functional groups, and can be connected to a plurality of different active groups, thereby avoiding the problems of limited connection sites of linear PEG, a small application range, and low drug loading. Moreover, the multi-arm polyethylene glycol-drug conjugate can also be used in the field of antibody-drug conjugates. Compared with a linear linker, a heterofunctional multi-arm polyethylene glycol linker can greatly increase the drug loading of a single antibody-drug conjugate molecule.

### TECHNICAL FIELD

The present disclosure relates to the technical field of pharmaceuticals, and particularly relates to a multi-arm polyethylene glycol-drug conjugate, a preparation method therefor, and use thereof.

### BACKGROUND

Polyethylene glycol and a derivative thereof have been widely used in biopharmaceuticals, pesticides, and medical materials due to their unique properties. Polyethylene glycol, with a well-defined metabolic process in the human body, is a safe and side effect-free synthetic polymer material. When conjugated with proteins, polypeptides, or drugs, polyethylene glycol can effectively prolong the physiological half-life of the conjugated drugs while reducing their immunogenicity and toxicity. In clinical applications, polyethylene glycol and a derivative thereof have been used as carriers for pharmaceutical formulations in many commercial pharmaceutical products. Meanwhile, the direct bonding of polyethylene glycol to drug molecules has achieved significant development over the past decade and has been applied in several approved drugs, such as the α-interferon-polyethylene glycol conjugate, which exhibits prolonged circulation half-life and enhanced therapeutic effect.

In drug modification, compared with linear polyethylene glycol, multi-arm polyethylene glycol has multiple terminal groups, thereby offering multiple drug conjugation sites and the advantage of loading multiple drug molecules. Currently, multi-arm polyethylene glycol is widely used for PEGylation modification of polypeptides and small molecule drugs.

Patent CN108472382A discloses an antibody-polymer conjugate comprising one or more anti-factor D antibody variants, a preparation method therefor, and use thereof. The conjugate comprises one or more anti-factor D antibody variants covalently linked to one or more polyols, i.e., all antibodies linked to the polyols have an identical structure; it does not disclose that the antibodies linked to the polyols are all PEGylated antibodies with different functional groups.

Patent WO2011075953A1 discloses a novel multi-arm polyethylene glycol formed by polymerizing ethylene oxide using oligomeric pentaerythritol as an initiator, which contains different types of active groups selected from: hydroxy, amino, sulfydryl, carboxyl, an ester group, an aldehyde group, an acrylic group, and a maleimide group;

it does not disclose that the active groups may be

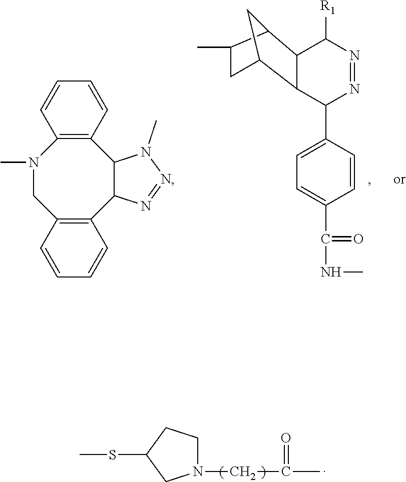

In the prior art, one or more antibodies linked to the central molecule (multi-arm polyethylene glycol) all have an identical structure, thereby failing to achieve simultaneous conjugation of two or even three different drugs within a single molecular system for the purpose of treating multiple diseases with one medicament. Additionally, the antibodies are not further modified by PEG. Furthermore, the structures of linkers linked to the antibodies are relatively common and homogeneous, which imposes certain limitations on effectively prolonging the drugs' physiological half-life, reducing their immunogenicity and toxicity, and the like.

### SUMMARY

To overcome the drawbacks existing in the prior art, the present disclosure provides a multi-arm polyethylene glycol-drug conjugate, a preparation method therefor, and use thereof. The conjugate uses multi-arm polyethylene glycol as a core, and three antibody drugs are simultaneously conjugated to one polyethylene glycol molecule, thereby achieving the purpose of treating multiple diseases with one medicament.

In a first aspect of the present disclosure, provided is a multi-arm polyethylene glycol-drug conjugate, which has a structure represented by general formula I:

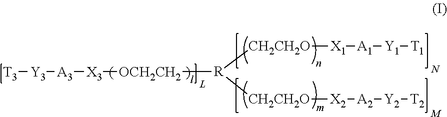

wherein R is a central molecule selected from one of a polyol residue, an oligopeptide residue, an amino acid residue, and an amino acid residue derivative.

Further, the polyol residue is selected from one of glycerol, polyglycerol, pentaerythritol, polypentaerythritol, a mannitol residue or a glycerol ether group thereof, methylglucoside, and sucrose.

In one embodiment of the present disclosure, the polyol residue is a glycerol residue.

Further, the amino acid residue has amino, carboxyl, and sulfydryl simultaneously.

Further, the amino acid residue is present in a quantity of one or two or more.

In one embodiment of the present disclosure, the amino acid residue is present in a quantity of one.

Further, when the amino acid residue is present in a quantity of two or more, the amino acid residues may be identical or different.

In one embodiment of the present disclosure, when the amino acid residue is present in a quantity of two or more, the amino acid residues are all different.

In one embodiment of the present disclosure, when the amino acid residue is present in a quantity of two or more, the amino acid residues are all identical.

In one embodiment of the present disclosure, when the amino acid residue is present in a quantity of two or more, the amino acid residues are partially identical.

In one embodiment of the present disclosure, the amino acid residue comprises one or two or more identical or different amino acid residues.

In one embodiment of the present disclosure, the central molecule comprises one amino acid residue.

In one embodiment of the present disclosure, the amino acid residue has a structure of:

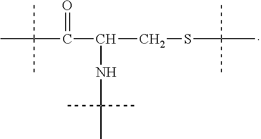

In one embodiment of the present disclosure, the central molecule is glycerol.

n, m, and 1 are each independently selected from an integer of 0-228; preferably, n, m, and 1 are each independently selected from an integer of 0-80; further preferably, n, m, and 1 are each independently selected from an integer of 0-60; and still further preferably, n, m, and 1 are each independently selected from an integer of 0-50.

In one embodiment of the present disclosure, n, m, and 1 are all mean values.

In one embodiment of the present disclosure, n, m, and 1 are all specified values.

In one embodiment of the present disclosure, n, m, and 1 are each independently an integer of 0.

In one embodiment of the present disclosure, n, m, and 1 are each independently an integer of 38.

X₁, X₂, and X₃ are all linking groups, and X₁, X₂, and X₃ are each independently selected from the group consisting of —(CH₂)ₐ—, —(CH₂)_{b}NH, —CO(CH₂)_{c}CO—, —(CH₂)_{d}CO—, —NH(CH₂)ₑNHCO—,

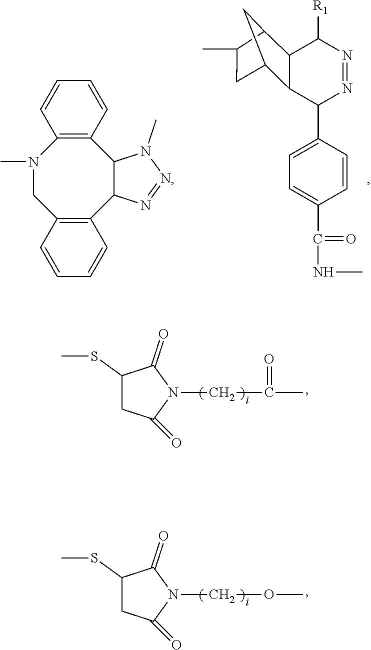

—NH—, —(CH₂)_{f}OCONH—, —CONH(CH₂)_{g}, —(CH₂)ₕNHCO—, —(CH₂)ⱼCONH—, and a combination thereof, wherein a, b, c, d, e, f, g, h, i, and j are each independently selected from an integer of 0-10 (e.g., 0, 1, 2, 3, 4, 5, 6, 7, 8, 9, and 10); R₁ is C₁₋₁₀ linear/branched alkyl; optionally, C atoms on X₁, X₂, and X₃ may be substituted with alkyl, alkoxy, amino, hydroxy, aryl, and heteroaryl.

In one embodiment of the present disclosure, R₁ is methyl.

Preferably, b is an integer selected from 0-5; and more preferably, b is 2.

Preferably, c is an integer selected from 0-5.

In one embodiment of the present disclosure, c is 1.

In one embodiment of the present disclosure, c is 2.

In one embodiment of the present disclosure, c is 4.

Preferably, a is an integer selected from 0-5; and more preferably, a is an integer selected from 0-3.

In one embodiment of the present disclosure, a is 1.

In one embodiment of the present disclosure, a is 2.

Preferably, i is an integer selected from 0-5; and more preferably, i is 2.

Preferably, j is an integer selected from 0-5; and more preferably, j is 2.

Preferably, g is an integer selected from 0-5; and more preferably, g is 0.

Preferably, h is an integer selected from 0-5; and more preferably, h is 2.

Further, X₂ is selected from the group consisting of —(CH₂)_{b}NH, —CO(CH₂)_{c}CO—, —NH—,

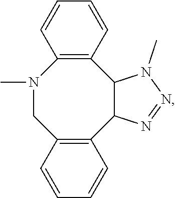 [SMILES: CN1Cc2ccccc2C2N=NN(C)C2c2ccccc21]

and a combination thereof.

In one embodiment of the present disclosure, X₂ is

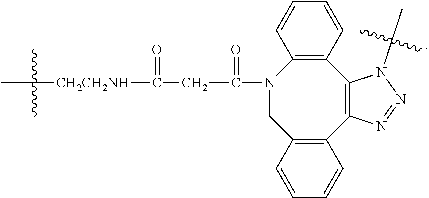

In one embodiment of the present disclosure, X₂ is

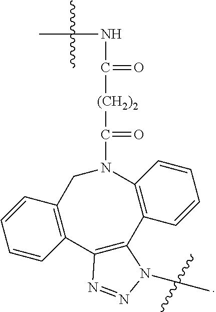

In one embodiment of the present disclosure, X₂ is

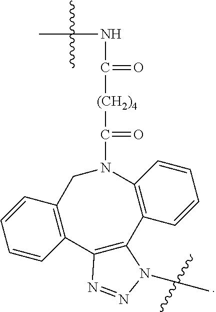

Further, X₃ is selected from the group consisting of —(CH₂)ₐ—,

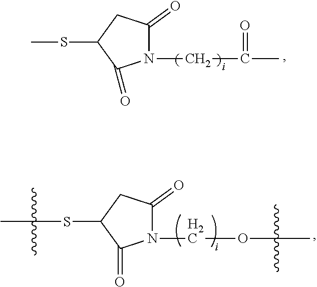

—NH—, and a combination thereof.

In one embodiment of the present disclosure, X₃ is

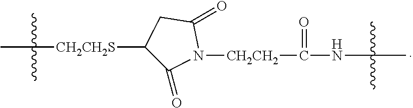

In one embodiment of the present disclosure, X₃ is

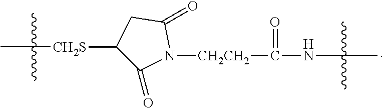

In one embodiment of the present disclosure, X₃ is

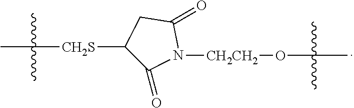

Further, X₁ is selected from the group consisting of —(CH₂)ⱼCONH—, —(CH₂)ₕNHCO—, —CONH(CH₂)_{g},

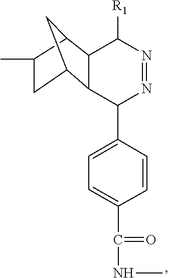

and a combination thereof.

In one embodiment of the present disclosure, X₁ is

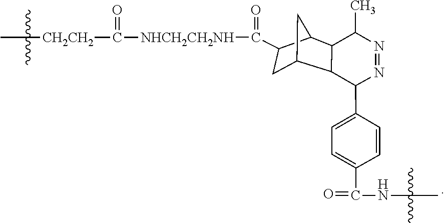

In one embodiment of the present disclosure, X₁ is

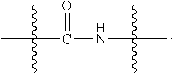 [SMILES: *NC(*)=O]

A₁, A₂, and A₃ are identical or different polyethylene glycol residues, separately.

Further, A₁, A₂, and A₃ comprise a —(CH₂CH₂O)ₚ— repeating unit.

Further, A₁, A₂, and A₃ are each independently selected from linear, Y-shaped, and multi-branched polyethylene glycol residues.

In one embodiment of the present disclosure, A₁, A₂, and A₃ are a linear polyethylene glycol residue, separately.

Further, P is an integer selected from 0-250 (e.g., 0, 10, 20, 30, 40, 44, 45, 46, 47, 50, 55, 56, 57, 60, 65, 66, 67, 68, 69, 70, 71, 75, 78, 79, 80, 81, 82, 85, 89, 90, 91, 92, 95, 100, 111, 112, 113, 114, 115, 116, 120, 150, 200, and 250); preferably, P is an integer selected from 0-114; further preferably, P is an integer selected from 0-80; still further preferably, P is an integer selected from 0-24; and particularly preferably, P is 12.

In one embodiment of the present disclosure, P is a mean value.

In one embodiment of the present disclosure, P is a specified value.

Further, A₁, A₂, and A₃ have a molecular weight of 0-11000 Da, separately (e.g., 0 Da, 500 Da, 600 Da, 700 Da, 800 Da, 900 Da, 1000 Da, 1100 Da, 1200 Da, 1300 Da, 1400 Da, 1500 Da, 1600 Da, 1700 Da, 1800 Da, 1900 Da, 2000 Da, 2500 Da, 3000 Da, 3100 Da, 3200 Da, 3300 Da, 3400 Da, 3500 Da, 3600 Da, 3700 Da, 3800 Da, 3900 Da, 4000 Da, 4500 Da, 5000 Da, 8000 Da, 9000 Da, 10000 Da, and 11000 Da); preferably, A₁, A₂, and A₃ have a molecular weight of 0-5000 Da, separately; further preferably, A₁, A₂, and A₃ have a molecular weight of 0-3500 Da, separately; and still further preferably, A₁, A₂, and A₃ have a molecular weight of 0-1100 Da, separately.

Y₁, Y₂, and Y₃ are all linking groups, and Y₁, Y₂, and Y₃ are each independently selected from the group consisting of —(CH₂)ₖ₁—, —NH—, —S—S—, —(CH₂)ₖ₂CO—, —(CH₂)ₖ₃ NHCO—,

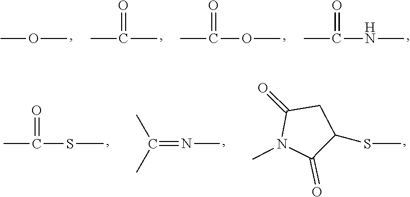

and a combination thereof, wherein K1, K2, and K3 are each independently selected from an integer of 1-10 (e.g., 1, 2, 3, 4, 5, 6, 7, 8, 9, and 10).

Preferably, K2 is an integer selected from 1-5; and further preferably, K2 is an integer selected from 1-3.

In one embodiment of the present disclosure, K2 is 1.

In one embodiment of the present disclosure, K2 is 2.

Further, Y₁, Y₂, and Y₃ are each independently selected from the group consisting of —(CH₂)ₖ₂CO—, —NH—, and a combination thereof.

In one embodiment of the present disclosure, Y₁, Y₂, and Y₃ are all —(CH₂)₂CO—NH—.

In one embodiment of the present disclosure, Y₁, Y₂, and Y₃ are all —(CH₂)CO—NH—.

T₁, T₂, and T₃ are each independently selected from an antibody, an antibody fragment, and a derivative thereof.

Preferably, the antibody is reactive to an antigen or an epitope thereof associated with cancer, a malignant cell, an infectious organism, or an autoimmune disease.

Further, T₁, T₂, and T₃ are each independently selected from one of a monoclonal antibody, a polyclonal antibody, a protein, and a polypeptide.

Further, T₁, T₂, and T₃ are each independently selected from an anti-CD3 antibody, an anti-EGFR antibody, an anti-CD28 antibody, an anti-HER2 antibody, an anti-PMSA antibody, an anti-VEGFR antibody, an anti-CD30 antibody, an anti-CD22 antibody, an anti-CD56 antibody, an anti-CD29 antibody, an anti-GPNMB antibody, an anti-CD138 antibody, an anti-CD74 antibody, an anti-ENPP3 antibody, an anti-Nectin-4 antibody, an anti-EGFRVIII antibody, an anti-SLC44A4 antibody, an anti-mesothelin antibody, an anti-ET8R antibody, an anti-CD37 antibody, an anti-CEACAM5 antibody, an anti-CD70 antibody, an anti-MUC16 antibody, an anti-CD79b antibody, an anti-MUC16 antibody, and an anti-Muc1 antibody.

Preferably, T₁, T₂, and T₃ are each independently selected from one of an anti-CD3 antibody, an anti-EGFR antibody, and an anti-CD28 antibody.

In one embodiment of the present disclosure, T₁ is an anti-EGFR antibody.

In one embodiment of the present disclosure, T₁ is an anti-CD3 antibody.

In one embodiment of the present disclosure, T₁ is an anti-CD28 antibody.

In one embodiment of the present disclosure, T₂ is an anti-EGFR antibody.

In one embodiment of the present disclosure, T₂ is an anti-CD3 antibody.

In one embodiment of the present disclosure, T₂ is an anti-CD28 antibody.

In one embodiment of the present disclosure, T₃ is an anti-EGFR antibody.

In one embodiment of the present disclosure, T₃ is an anti-CD3 antibody.

In one embodiment of the present disclosure, T₃ is an anti-CD28 antibody.

N, M, and L are each independently selected from an integer of 1-24 (e.g., 1, 2, 3, 4, 5, 6, 7, 8, 9, 10, 11, 12, 13, 14, 15, 16, 17, 18, 19, 20, 21, 22, 23, and 24), and N+M+L≥3.

In one embodiment of the present disclosure, N, M, and L are all 1.

Further, the multi-arm polyethylene glycol-drug conjugate has a structure represented by general formula II or III:

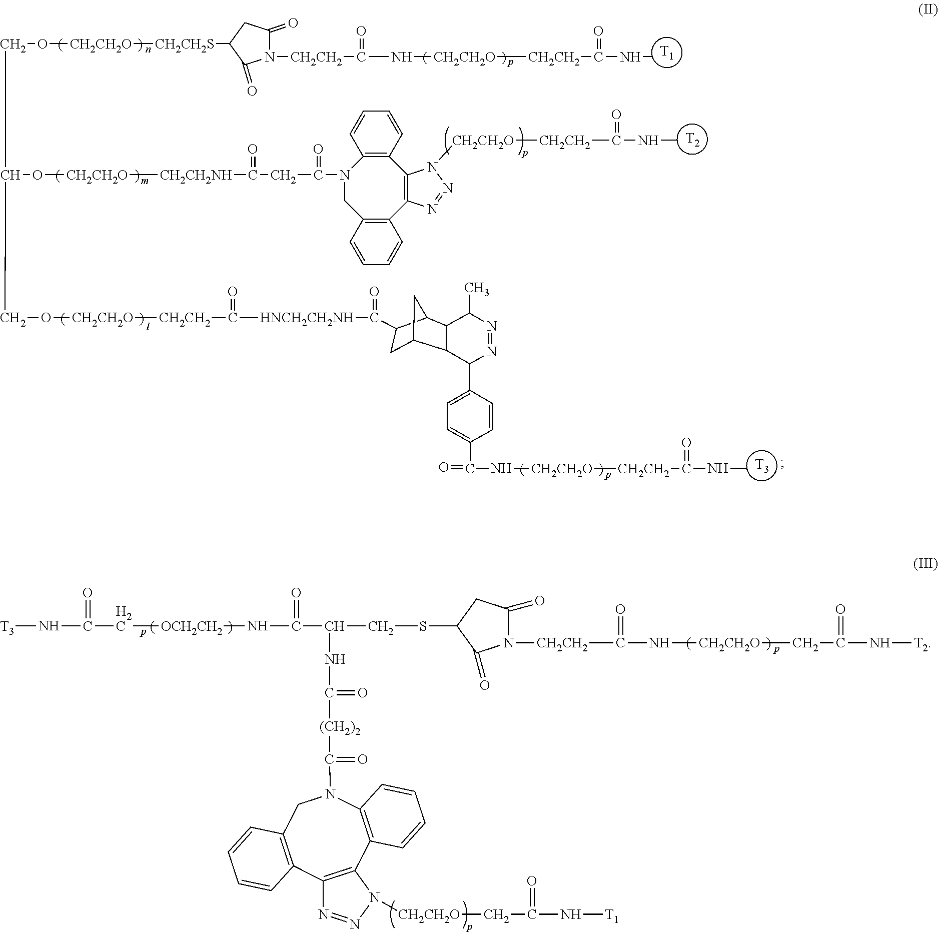

In one embodiment of the present disclosure, the multi-arm polyethylene glycol-drug conjugate has a structure represented by formula IV:

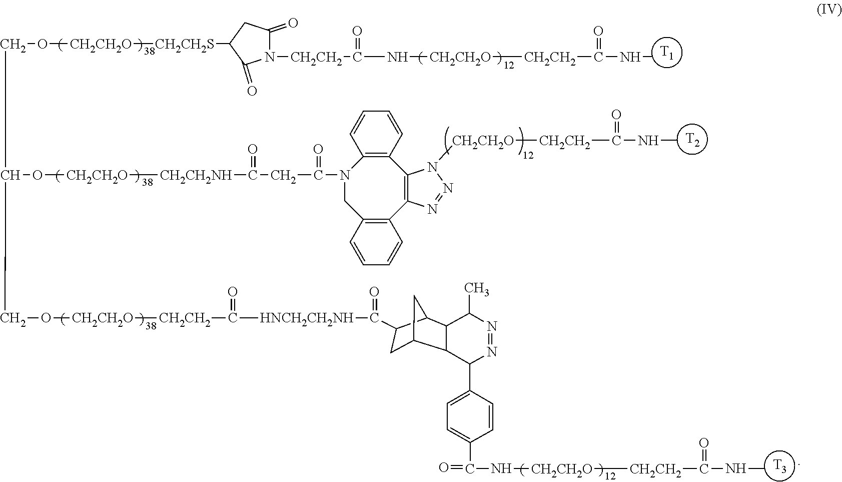

In one embodiment of the present disclosure, the multi-arm polyethylene glycol-drug conjugate has a structure represented by formula V:

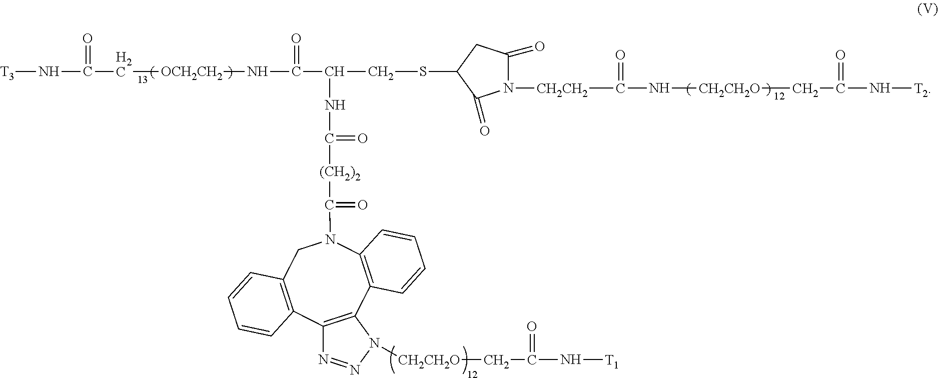

In one embodiment of the present disclosure, the multi-arm polyethylene glycol-drug conjugate has a structure represented by formula VI:

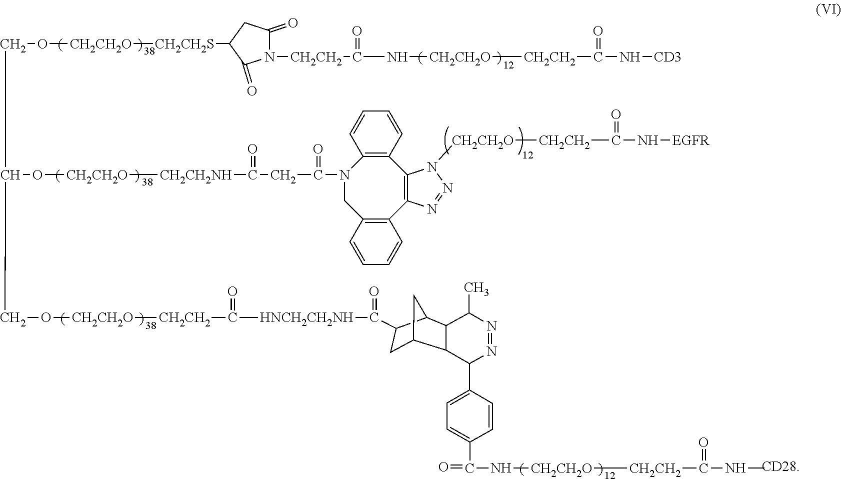

In one embodiment of the present disclosure, the multi-arm polyethylene glycol-drug conjugate has a structure represented by formula VII:

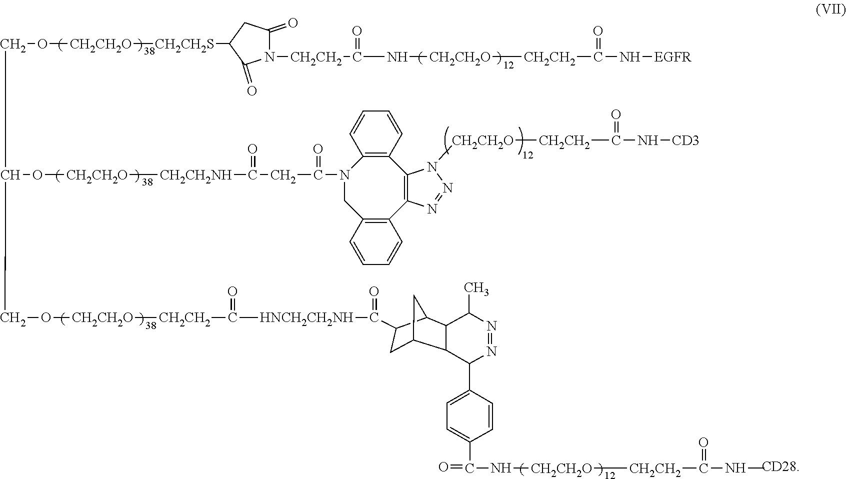

In one embodiment of the present disclosure, the multi-arm polyethylene glycol-drug conjugate has a structure represented by formula VIII:

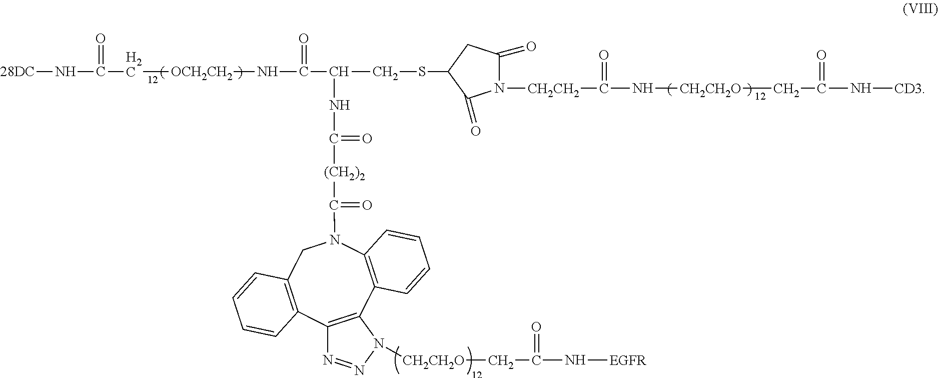

In one embodiment of the present disclosure, the multi-arm polyethylene glycol-drug conjugate has a structure represented by formula IX:

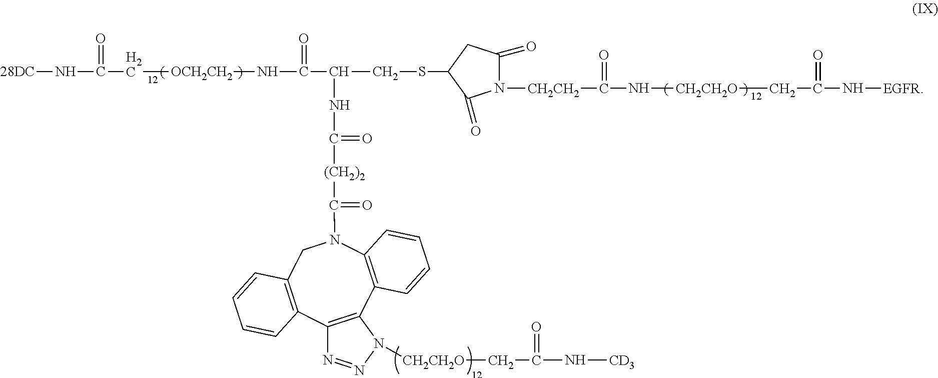

In a second aspect of the present disclosure, provided is a preparation method for the multi-arm polyethylene glycol-drug conjugate according to the first aspect, which comprises the following steps:
first, synthesizing a trifunctional polyethylene glycol derivative and PEG-modified antibodies with different functional groups, separately, and subsequently, reacting the two to synthesize the multi-arm polyethylene glycol-drug conjugate.

Further, the trifunctional polyethylene glycol derivative has a structure represented by general formula X:

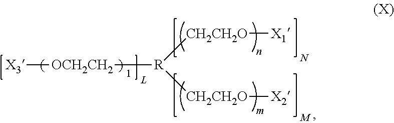

further, the PEG-modified antibodies with different functional groups are designated as MAL-A₁-Y₁-T₁, MTZ-A₂-Y₂-T₂, and N3-A₃-Y₃-T₃, respectively, wherein R is a central molecule selected from one of a polyol residue, an oligopeptide residue, an amino acid residue, and an amino acid residue derivative.

Further, the polyol residue is selected from one of glycerol, polyglycerol, pentaerythritol, polypentaerythritol, a mannitol residue or a glycerol ether group thereof, methylglucoside, and sucrose.

In one embodiment of the present disclosure, the polyol residue is a glycerol residue.

Further, the amino acid residue has amino, carboxyl, and sulfydryl simultaneously.

Further, the amino acid residue is present in a quantity of one or two or more.

In one embodiment of the present disclosure, the amino acid residue is present in a quantity of one.

Further, when the amino acid residue is present in a quantity of two or more, the amino acid residues may be identical or different.

In one embodiment of the present disclosure, when the amino acid residue is present in a quantity of two or more, the amino acid residues are all different.

In one embodiment of the present disclosure, when the amino acid residue is present in a quantity of two or more, the amino acid residues are all identical.

In one embodiment of the present disclosure, when the amino acid residue is present in a quantity of two or more, the amino acid residues are partially identical.

In one embodiment of the present disclosure, the amino acid residue comprises one or two or more identical or different amino acid residues.

In one embodiment of the present disclosure, the central molecule comprises one amino acid residue.

In one embodiment of the present disclosure, the amino acid residue has a structure of:

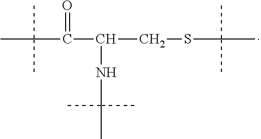

In one embodiment of the present disclosure, the central molecule is glycerol.

n, m, and 1 are each independently selected from an integer of 0-228; preferably, n, m, and 1 are each independently selected from an integer of 0-80; further preferably, n, m, and 1 are each independently selected from an integer of 0-60; and still further preferably, n, m, and 1 are each independently selected from an integer of 0-50.

In one embodiment of the present disclosure, n, m, and 1 are all mean values.

In one embodiment of the present disclosure, n, m, and 1 are all specified values.

In one embodiment of the present disclosure, n, m, and 1 are each independently an integer of 0.

In one embodiment of the present disclosure, n, m, and 1 are each independently an integer of 38.

X₁′, X₂′, and X₃′ are all linking groups.

X₁′, X₂′, and X₃′ are different from each other.

X₁′, X₂′, and X₃′ are each independently selected from the group consisting of

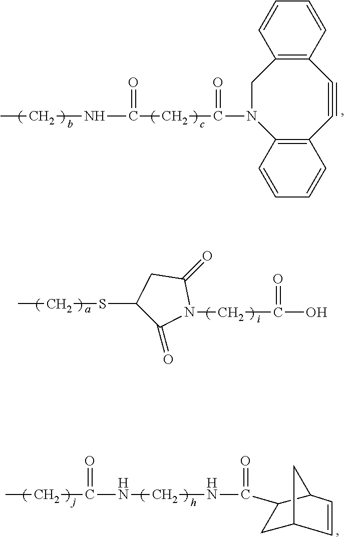

—(CH₂)_{b}NH₂, —CO(CH₂)_{c}COOH, —(CH₂)_{d}COOH, —NH(CH₂)ₑNHCOOH,

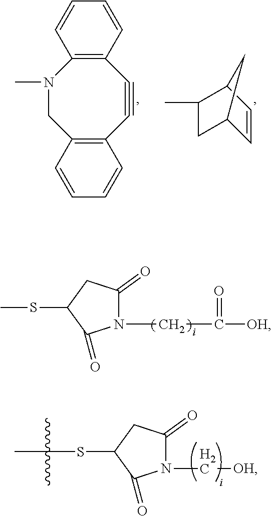

—NH₂, —(CH₂)_{f}OCONH₂, —CONH(CH₂)_{g}—CH₃, —(CH₂)ₕNHCOOH, —(CH₂)ⱼCONH₂, and a combination thereof, wherein a, b, c, d, e, f, g, h, i, and j are each independently selected from an integer of 0-10, and R₁ is C₁₋₁₀ linear/branched alkyl; optionally, C atoms on X₁′, X₂′, and X₃′ are substituted with alkyl, alkoxy, amino, hydroxy, aryl, and heteroaryl; preferably, R₁ is methyl,
wherein a, b, c, d, e, f, g, h, i, and j are each independently selected from an integer of 0-10 (e.g., 0, 1, 2, 3, 4, 5, 6, 7, 8, 9, and 10).

Preferably, b is an integer selected from 0-5; and more preferably, b is 2.

Preferably, c is an integer selected from 0-5.

In one embodiment of the present disclosure, c is 1.

In one embodiment of the present disclosure, c is 2.

In one embodiment of the present disclosure, c is 4.

Preferably, a is an integer selected from 0-5; and more preferably, a is an integer selected from 0-3.

In one embodiment of the present disclosure, a is 1.

In one embodiment of the present disclosure, a is 2.

Preferably, i is an integer selected from 0-5; and more preferably, i is 2.

Preferably, j is an integer selected from 0-5; and more preferably, j is 2.

Preferably, g is an integer selected from 0-5; and more preferably, g is 0.

Preferably, h is an integer selected from 0-5; and more preferably, h is 2.

N, M, and L are each independently selected from an integer of 1-24 (e.g., 1, 2, 3, 4, 5, 6, 7, 8, 9, 10, 11, 12, 13, 14, 15, 16, 17, 18, 19, 20, 21, 22, 23, and 24), and N+M+L≥3.

In one embodiment of the present disclosure, N, M, and L are all 1.

Further, A₁, A₂, and A₃ are identical or different polyethylene glycol residues, separately.

Further, A₁, A₂, and A₃ comprise a —(CH₂CH₂O)ₚ— repeating unit.

Further, A₁, A₂, and A₃ are each independently selected from linear, Y-shaped, and multi-branched polyethylene glycol residues.

In one embodiment of the present disclosure, A₁, A₂, and A₃ are a linear polyethylene glycol residue, separately.

Further, P is an integer selected from 0-250 (e.g., 0, 10, 20, 30, 40, 44, 45, 46, 47, 50, 55, 56, 57, 60, 65, 66, 67, 68, 69, 70, 71, 75, 78, 79, 80, 81, 82, 85, 89, 90, 91, 92, 95, 100, 111, 112, 113, 114, 115, 116, 120, 150, 200, and 250); preferably, P is an integer selected from 0-114; further preferably, P is an integer selected from 0-80; still further preferably, P is an integer selected from 0-24; and particularly preferably, P is 12.

In one embodiment of the present disclosure, P is a mean value.

In one embodiment of the present disclosure, P is a specified value.

Further, A₁, A₂, and A₃ have a molecular weight of 0-11000 Da, separately (e.g., 0 Da, 500 Da, 600 Da, 700 Da, 800 Da, 900 Da, 1000 Da, 1100 Da, 1200 Da, 1300 Da, 1400 Da, 1500 Da, 1600 Da, 1700 Da, 1800 Da, 1900 Da, 2000 Da, 2500 Da, 3000 Da, 3100 Da, 3200 Da, 3300 Da, 3400 Da, 3500 Da, 3600 Da, 3700 Da, 3800 Da, 3900 Da, 4000 Da, 4500 Da, 5000 Da, 8000 Da, 9000 Da, 10000 Da, and 11000 Da); preferably, A₁, A₂, and A₃ have a molecular weight of 0-5000 Da, separately; further preferably, A₁, A₂, and A₃ have a molecular weight of 0-3500 Da, separately; and still further preferably, A₁, A₂, and A₃ have a molecular weight of 0-1100 Da, separately. Y₁, Y₂, and Y₃ are all linking groups, and Y₁, Y₂, and Y₃ are each independently selected from the group consisting of —(CH₂)ₖ₁—, —NH—, —S—S—, —(CH₂)ₖ₂CO—, —(CH₂)ₖ₃ NHCO—,

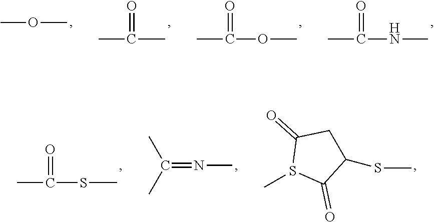

and a combination thereof, wherein K1, K2, and K3 are each independently selected from an integer of 1-10 (e.g., 1, 2, 3, 4, 5, 6, 7, 8, 9, and 10).

Preferably, K2 is an integer selected from 1-5; and further preferably, K2 is an integer selected from 1-3.

In one embodiment of the present disclosure, K2 is 1.

In one embodiment of the present disclosure, K2 is 2.

Further, Y₁, Y₂, and Y₃ are each independently selected from the group consisting of —(CH₂)ₖ₂CO—, —NH—, and a combination thereof.

In one embodiment of the present disclosure, Y₁, Y₂, and Y₃ are all —(CH₂)₂CO—NH—.

In one embodiment of the present disclosure, Y₁, Y₂, and Y₃ are all —(CH₂)CO—NH—.

T₁, T₂, and T₃ are each independently selected from an antibody, an antibody fragment, and a derivative thereof.

Preferably, the antibody is reactive to an antigen or an epitope thereof associated with cancer, a malignant cell, an infectious organism, or an autoimmune disease.

Further, T₁, T₂, and T₃ are each independently selected from one of a monoclonal antibody, a polyclonal antibody, a protein, and a polypeptide.

Further, T₁, T₂, and T₃ are each independently selected from an anti-CD3 antibody, an anti-EGFR antibody, an anti-CD28 antibody, an anti-HER2 antibody, an anti-PMSA antibody, an anti-VEGFR antibody, an anti-CD30 antibody, an anti-CD22 antibody, an anti-CD56 antibody, an anti-CD29 antibody, an anti-GPNMB antibody, an anti-CD138 antibody, an anti-CD74 antibody, an anti-ENPP3 antibody, an anti-Nectin-4 antibody, an anti-EGFRVIII antibody, an anti-SLC44A4 antibody, an anti-mesothelin antibody, an anti-ET8R antibody, an anti-CD37 antibody, an anti-CEACAM5 antibody, an anti-CD70 antibody, an anti-MUC16 antibody, an anti-CD79b antibody, an anti-MUC16 antibody, and an anti-Muc1 antibody.

Preferably, T₁, T₂, and T₃ are each independently selected from one of an anti-CD3 antibody, an anti-EGFR antibody, and an anti-CD28 antibody.

In one embodiment of the present disclosure, T₁ is an anti-EGFR antibody.

In one embodiment of the present disclosure, T₁ is an anti-CD3 antibody.

In one embodiment of the present disclosure, T₁ is an anti-CD28 antibody.

In one embodiment of the present disclosure, T₂ is an anti-EGFR antibody.

In one embodiment of the present disclosure, T₂ is an anti-CD3 antibody.

In one embodiment of the present disclosure, T₂ is an anti-CD28 antibody.

In one embodiment of the present disclosure, T₃ is an anti-EGFR antibody.

In one embodiment of the present disclosure, T₃ is an anti-CD3 antibody.

In one embodiment of the present disclosure, T₃ is an anti-CD28 antibody.

Preferably, MAL-A₁-Y₁-T₁ has a structure of:

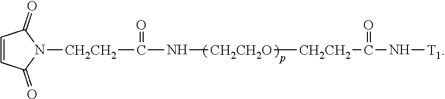

Preferably, MTZ-A₂-Y₂-T₂ has a structure of:

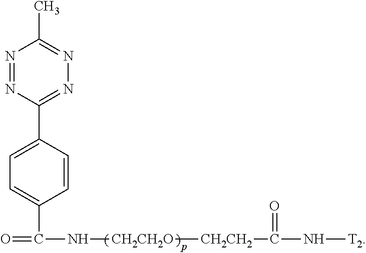

Preferably, N3-A₃-Y₃-T₃ has a structure of:

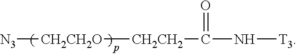

Preferably, the trifunctional polyethylene glycol derivative has a structure represented by general formula XI or XII:

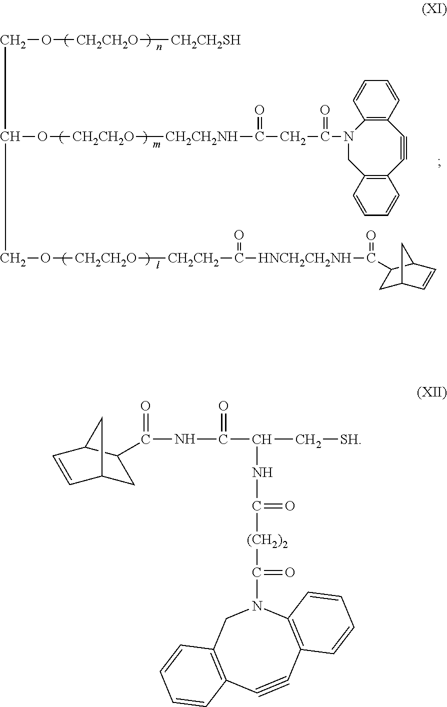

Further, the preparation method further comprises a separation and purification step.

In one embodiment of the present disclosure, the separation and purification refers to separating and purifying a trifunctional polyethylene glycol derivative (e.g., 3ARM-DBCO-SH-Norbornene PEG).

In one embodiment of the present disclosure, the separation and purification refers to separating and purifying MAL-A₁-Y₁-T₁.

In one embodiment of the present disclosure, the separation and purification refers to separating and purifying MTZ-A₂-Y₂-T₂.

In one embodiment of the present disclosure, the separation and purification refers to separating and purifying N3-A₃-Y₃-T₃.

In one embodiment of the present disclosure, the separation and purification refers to separating and purifying a multi-arm polyethylene glycol-drug conjugate.

In a fourth aspect of the present disclosure, provided is a pharmaceutical composition comprising a multi-arm polyethylene glycol-drug conjugate of general formula I, II, III, IV, V, VI, VII, VIII, or IX.

Preferably, the pharmaceutical composition further comprises one or more pharmaceutically acceptable auxiliary materials selected from: a carrier, a diluent, a binder, a lubricant, and a wetting agent.

Preferably, a dosage form of the pharmaceutical composition includes: a tablet, a capsule, a pill, an injection, an emulsion, a microemulsion, a nanoparticle, an inhalant, a lozenge, a gel, a powder, a suppository, a suspoemulsion, a cream, a jelly, and a spray. Preferably, the pharmaceutical composition may be administered in a mode including: oral administration, subcutaneous injection, intramuscular injection, intravenous injection, rectal administration, vaginal administration, nasal administration, transdermal administration, subconjunctival administration, intraocular administration, orbital administration, retrobulbar administration, retinal administration, choroidal administration, and intrathecal injection.

In a fifth aspect of the present disclosure, provided is use of the multi-arm polyethylene glycol-drug conjugate according to the first aspect or the pharmaceutical composition according to the third aspect in preparing a targeted drug for preventing and/or treating a disease; preferably, the disease is selected from a tumor, an infectious disease, hyperlipidemia, cancer, an autoimmune disease, an inflammatory disease, a neurodegenerative disease, an ocular disease, a rare disease, and the like.

The term “cancer” described in the present disclosure includes, but is not limited to, lymphoma, B cell tumor, T cell tumor, myeloid/monocytic tumor, non-small cell lung cancer, leukemia, ovarian cancer, nasopharyngeal cancer, breast cancer, endometrial cancer, colon cancer, rectal cancer, stomach cancer, bladder cancer, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myelodysplastic syndrome, and sarcoma, wherein the leukemia is selected from acute lymphocytic (lymphoblastic) leukemia, acute myelocytic leukemia, myeloid leukemia, chronic lymphocytic leukemia, multiple myeloma, plasma cell leukemia, and chronic myelocytic leukemia; the lymphoma is selected from Hodgkin lymphoma and non-Hodgkin lymphoma, including B-cell lymphoma, diffuse large B-cell lymphoma, follicular lymphoma, mantle cell lymphoma, marginal zone B-cell lymphoma, T-cell lymphoma, and Waldenstrom's macroglobulinemia; and the sarcoma is selected from osteosarcoma, Ewing's sarcoma, leiomyosarcoma, synovial sarcoma, soft tissue sarcoma, angiosarcoma, liposarcoma, fibrosarcoma, rhabdomyosarcoma, and chondrosarcoma.

The term “autoimmune disease” described in the present disclosure includes, but is not limited to, allergy, asthma, myocarditis, nephritis, hepatitis, systemic lupus erythematosus, rheumatoid arthritis, scleroderma, hyperthyroidism, idiopathic thrombocytopenic purpura, autoimmune hemolytic anemia, ulcerative colitis, autoimmune liver disease, diabetes, myasthenia gravis, multiple sclerosis, urticaria, psoriasis, dermatomyositis, Sjogren's syndrome, pain, neurological disorder, and the like.

The term “inflammatory disease” described in the present disclosure includes acute inflammation and chronic inflammation. Specifically, it includes, but is not limited to, alterative inflammation, exudative inflammation, proliferative inflammation, specific inflammation, and the like, including but not limited to severe burns, endotoxemia, septic shock, adult respiratory distress syndrome, hemodialysis, anaphylactic shock, severe asthma, angioedema, Crohn's disease, sickle cell anemia, post-streptococcal glomerulonephritis, pancreatitis, enteritis, vasculitis, adverse drug reactions, drug allergies, IL-2-induced vascular leak syndrome, radiographic contrast media allergies, and the like.

The term “neurodegenerative disease” described in the present disclosure includes, but is not limited to, Alzheimer's disease, progressive blindness or external ophthalmoplegia, multiple system atrophy, frontotemporal dementia, Huntington's chorea, corticobasal degeneration, spinocerebellar ataxia, motor neuron disease, hereditary motor sensory neuropathy, and the like.

The term “ocular disease” described in the present disclosure includes, but is not limited to, macular degenerative diseases such as all stages of age-related macular degeneration (AMD) including dry and wet (non-exudative and exudative) forms, diabetic retinopathy and other ischemia-related retinopathies, choroidal neovascularization (CNV), uveitis, diabetic macular edema, pathological myopia, von Hippel-Lindau disease, ocular histoplasmosis, central retinal vein occlusion (CRVO), corneal neovascularization, and retinal neovascularization. Age-related macular degeneration (AMD) includes non-exudative (e.g. intermediate dry AMD or geographic atrophy (GA)) and exudative (e.g. wet AMD (choroidal neovascularization (CNV))) AMD, diabetic retinopathy (DR), endophthalmitis, and uveitis. In addition, the non-exudative AMD may include hard drusen, soft drusen, geographic atrophy and/or pigment agglomeration, and the like.

The multi-arm polyethylene glycol-drug conjugates represented by general formulas I, II, III, IV, V, VI, and VII provided by the present disclosure, compared with linear polyethylene glycol-drug conjugates, feature a multi-arm PEG structure with multiple terminal groups that provide multiple sites for introducing functional groups. This allows the conjugation of diverse active groups, thereby overcoming the limitations of linear PEG such as restricted conjugation sites, narrow application scope, and low drug loading capacity. Additionally, the multi-arm polyethylene glycol-drug conjugates provided by the present disclosure are characterized in that the terminal positions of the multi-arm polyethylene glycol structure may be heterofunctional groups, thereby achieving the simultaneous conjugation of two or even three drugs within a single molecular system for the purpose of treating multiple diseases with one medicament.

Moreover, the multi-arm polyethylene glycol-drug conjugates can also be used in the field of antibody-drug conjugates. Compared with a linear linker, a heterofunctional multi-arm polyethylene glycol linker can greatly increase the drug loading of a single antibody-drug conjugate molecule.

Unless otherwise defined, all scientific and technical terms used in the present disclosure have the same meaning as commonly understood by those skilled in the art to which the present disclosure relates.

The term C1-10 linear/branched alkyl described in the present disclosure includes methyl, ethyl, C3 linear/branched alkyl, C4 linear/branched alkyl, C5 linear/branched alkyl, C6 linear/branched alkyl, C7 linear/branched alkyl, C8 linear/branched alkyl, C9 linear/branched alkyl, and C10 linear/branched alkyl.

The term “norbornene” described in the present disclosure has a structure of

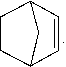

The term “OPA” described in the present disclosure refers to a propionic acid residue having a structure of

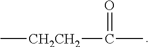

The term “SOPSS” described in the present disclosure refers to 2-mercaptopyridine having a structure of

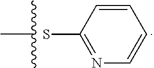

The term “S-MAL” refers to sulfydryl-maleimide having a structure of

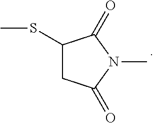

The term “DBCO” described in the present disclosure refers to dibenzocyclooctyne having a structure of

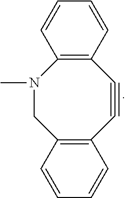 [SMILES: CN1Cc2ccccc2C#Cc2ccccc21]

The term “Me” described in the present disclosure refers to methyl having a structure of —CH₃.

The term “t-BuOK” described in the present disclosure refers to potassium tert-butoxide.

The term “THF” described in the present disclosure refers to tetrahydrofuran.

The term “MsCl” described in the present disclosure refers to methanesulfonyl chloride.

The term “TEA” described in the present disclosure refers to triethylamine.

The term “DCM” described in the present disclosure refers to dichloromethane.

The term “NHS” described in the present disclosure refers to N-hydroxysulfosuccinimide.

The term “DCC” described in the present disclosure refers to dicyclohexylcarbodiimide.

The term “antibody” described in the present disclosure is used in its broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al., (2003), Jour. of Immunology, 170:4854-4861). The antibody may be a murine antibody, a human antibody, a humanized antibody, or a chimeric antibody, or derived from other species. The antibody is a protein produced by an immune system that is capable of recognizing and binding to a specific antigen (Janeway, C. et al., (2001), ImmunoBiology, 5th Ed., Garland Publishing, New York).

The term “monoclonal antibody” described in the present disclosure refers to an antibody obtained from a population of substantially homogeneous antibodies, i.e., each antibody contained in the population is identical except for possible naturally-occurring mutations that may be present in minor amounts. Monoclonal antibodies are highly specific antibodies that target a single antigenic site. Moreover, in contrast to polyclonal antibody preparations that typically include different antibodies targeting different determinants (epitopes), each monoclonal antibody targets only a single determinant on the antigen. In addition to their specificity, the monoclonal antibodies are also advantageous in that they can be synthesized in a manner that is uncontaminated by other antibodies. The modifier “monoclonal” indicates the characteristic of the antibody obtained from a population of substantially homogeneous antibodies, and is not to be construed as requiring production of the antibody by any particular method.

The term “polyethylene glycol” described in the present disclosure represents a polymer comprising a (CH₂CH₂O) repeating group.

The term “treating” described in the present disclosure includes eradicating, removing, reversing, alleviating, altering, or controlling a disease and/or condition after its onset.

The term “preventing” described in the present disclosure refers to the ability to avoid, minimize, or hinder the onset or progression of a disease and/or condition prior to its onset through treatment.

The term “disease” described in the present disclosure refers to a physical condition of the subject that is associated with the disease and/or condition of the present disclosure.

### BRIEF DESCRIPTION OF THE DRAWINGS

FIG. 1 is the nuclear magnetic resonance spectrum of intermediate 3.

FIG. 2 is the nuclear magnetic resonance spectrum of intermediate 4.

FIG. 3 is the nuclear magnetic resonance spectrum of intermediate 5.

FIG. 4 is the nuclear magnetic resonance spectrum of intermediate 6.

FIG. 5 is the nuclear magnetic resonance spectrum of intermediate 8.

FIG. 6 is the nuclear magnetic resonance spectrum of intermediate 11.

FIG. 7 is the nuclear magnetic resonance spectrum of intermediate 12.

FIG. 8 is the nuclear magnetic resonance spectrum of norbornene-2-formamidoethylamine.

FIG. 9 is the nuclear magnetic resonance spectrum of intermediate 13.

FIG. 10 is the nuclear magnetic resonance spectrum of intermediate 14.

FIG. 11 is the nuclear magnetic resonance spectrum of three-arm polyethylene glycol-monopropionylaminoethylaminenorbornene-monoamino DBCO-monothiol-2-thiopyridine.

FIG. 12 is the schematic diagram of purity analysis results for the trispecific antibody formed via PEG conjugation.

FIG. 13 is the schematic diagram of the binding ability of the EGFR monoclonal antibody and the trispecific antibody to target cells detected by flow cytometry (with the EGFR monoclonal antibody on the left and the trispecific antibody on the right).

FIG. 14 is the schematic diagram of antibody-mediated T cell killing activity against target cells detected by the CCK8 assay.

FIG. 15 is the schematic diagram of IL-2 secretion levels detected by the ELISA assay.

### DETAILED DESCRIPTION

The technical solutions of the present disclosure will be clearly and completely described below with reference to the examples of the present disclosure, and it is obvious that the described examples are only a part of the examples of the present disclosure but not all of them. Based on the examples of the present disclosure, all other examples obtained by those of ordinary skill in the art without creative work shall fall within the protection scope of the present disclosure.

The term “N3” refers to an azide group.

The term “TCO” refers to a trans-cyclooctene group.

The term “Tetrazine” and the term “MTZ” both refer to a tetrazine group.

The term “MAL” refers to a maleimide group.

### EXAMPLES

Various embodiments of the present disclosure are illustrated by the following examples, but are not intended to limit the present disclosure.

### Example 1

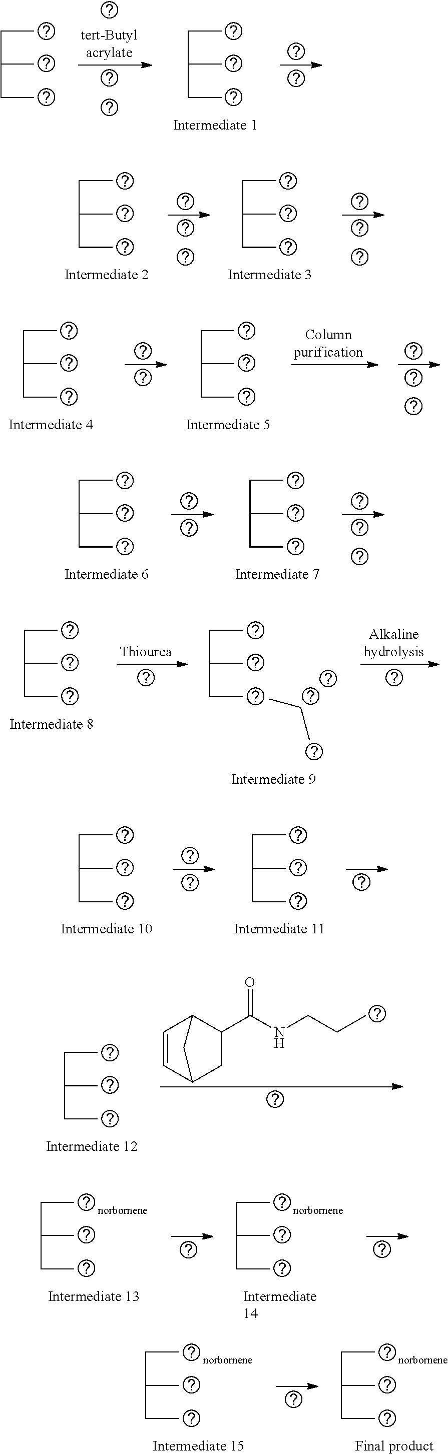

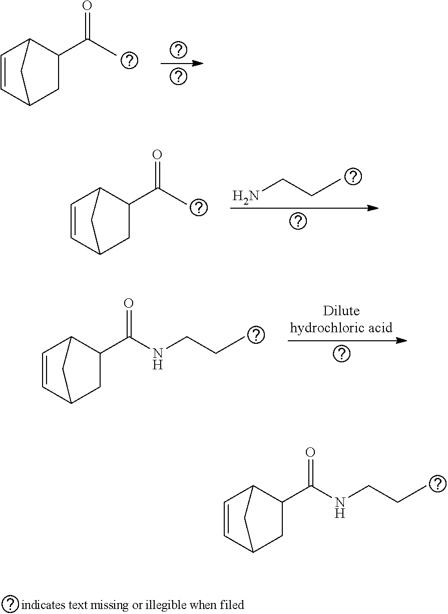

### 1.1: Synthesis of Three-Arm Polyethylene glycol-monopropionic acid

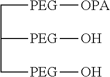

### Intermediate 2

Procedure: A three-necked round bottom flask was purged with nitrogen, and 500 g of three-arm polyethylene glycol-5K and 3 L of toluene were added. The mixture was heated for dissolving, and about two-thirds of toluene was distilled off. After cooling, 3 L of THE and 2.8 g of potassium tert-butoxide were added, and the mixture was allowed to react at room temperature for 2 h. 21.8 g of tert-butyl acrylate was added dropwise, and the resulting mixture was allowed to react at room temperature overnight and filtered the next day. The reaction solution was concentrated under vacuum to give a viscous liquid. 3 M dilute hydrochloric acid (prepared by 4 Vol. dilution of concentrated hydrochloric acid) was added to the system described above. Acidolysis was performed for 4-5 h. 15% sodium chloride was added to the solution, and the mixture was extracted three times with dichloromethane. The organic phases were combined, dried over anhydrous sodium sulfate, filtered, and concentrated to a viscous state. 500 g of the crude product was separated and purified by DEAE anion exchange column chromatography. The sodium chloride eluate was collected. The pH was adjusted to 2-3 with 2 N dilute hydrochloric acid, and the mixture was extracted with dichloromethane. The organic phases were combined, dried over anhydrous sodium sulfate, allowed to stand overnight, and subjected to hot dissolution and cold crystallization in 10.0 L of an isopropanol-ethyl acetate (3:1) mixed solvent to give three-arm polyethylene glycol monopropionic acid with a molecular weight of 5000.

NMR (DMSO) 2.45 (t, CH₂COOCH₃) 3.7 (t, CH₂—CH₂COOCH₃)

### 1.2: Synthesis of Three-Arm polyethylene glycol-methyl monopropionate

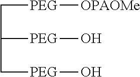

Intermediate 3 Procedure: A three-necked round bottom flask was purged with nitrogen, and 100 g of three-arm polyethylene glycol monopropionic acid-5000 and 1 L of anhydrous methanol were added. 20 mL of concentrated sulfuric acid was slowly added dropwise, and the mixture was allowed to react at room temperature for 3 h. The pH of the system was adjusted to about 7.0 with an 8% aqueous sodium bicarbonate solution, and the mixture was extracted three times with dichloromethane. The organic phases were combined, dried over anhydrous sodium sulfate, filtered under vacuum, concentrated, subjected to hot dissolution and cold crystallization in 2.0 L of an isopropanol-ethyl acetate (3:1) mixed solvent, and dried under vacuum to give three-arm polyethylene glycol methyl monopropionate.

NMR (DMSO) 2.45 (t, CH₂COOCH₃), 3.32 (S, CH₂COOCH₃), 3.7 (t, CH₂—CH₂COOCH₃)

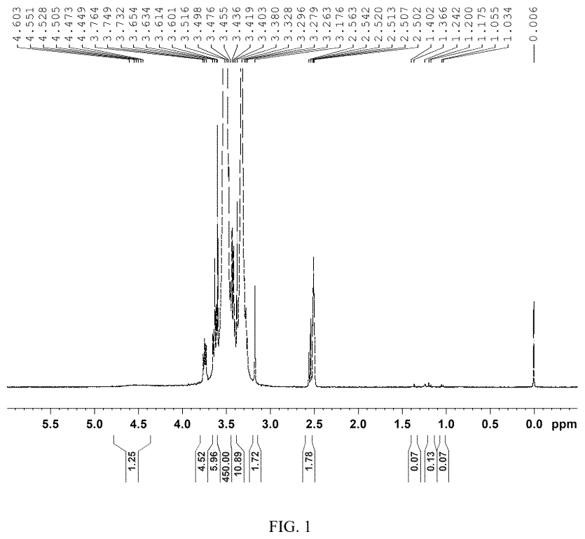

The nuclear magnetic resonance spectrum of intermediate 3 is shown in FIG. 1.

### 1.3: Synthesis of Three-Arm polyethylene glycol-methyl monopropionate mono-methanesulfonate

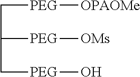

### Intermediate 4

Procedure: A three-necked round bottom flask was purged with nitrogen, and 90 g of three-arm polyethylene glycol methyl monopropionate, 500 mL of dichloromethane, 2.51 g of methanesulfonyl chloride (MsCl), and 3.10 g of triethylamine were added. The reaction system was sealed and allowed to stand overnight. The next day, 0.2 mL of ethanol was added, and the mixture was stirred for 15 min and concentrated to a viscous state. The resulting mixture was subjected to hot dissolution and cold crystallization in 1.8 L of an isopropanol-ethyl acetate (3:1) mixed solvent and dried under vacuum to give three-arm polyethylene glycol methyl monopropionate monomethanesulfonate.

NMR (DMSO) 2.45 (t, CH₂COOCH₃) 3.17 (S, SO₂CH₃), 3.7 (t, CH₂—CH₂COOCH₃), 4.32 ppm (t, CH₂OOSO₂CH₃)

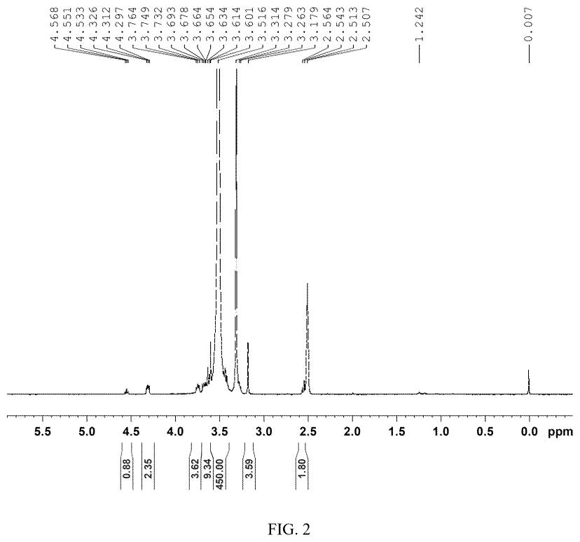

The nuclear magnetic resonance spectrum of intermediate 4 is shown in FIG. 2.

### 1.4: Synthesis of Three-Arm polyethylene glycol-monopropionic acid-monoamine

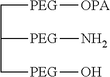

### Intermediate 5

Procedure: A three-necked round bottom flask was purged with nitrogen, and 80 g of three-arm polyethylene glycol methyl monopropionate monomethanesulfonate was added and dissolved in 400 mL of degassed water. The pH was adjusted to about 12 with a 2 N sodium hydroxide solution, and the mixture was allowed to react at room temperature for 3 h. 1 L of an aqueous ammonia solution containing 50 g of ammonium chloride was added to the system. The mixture was stirred at room temperature for 72 h. After completion of the reaction, 300 g of sodium chloride was added, and the mixture was extracted three times with dichloromethane. The organic phases were combined and concentrated to dryness at 40° C. The residue was dissolved in 1 L of degassed water until clear. The pH was adjusted to 2-3 with 2 N dilute hydrochloric acid, and 200 g of sodium chloride was added. The mixture was extracted three times with dichloromethane again. The organic phases were combined, dried over anhydrous sodium sulfate, and concentrated to dryness at 40° C. The residue was purified by CM-DEAE ion exchange column chromatography. The water-eluted fractions were collected. The pH was adjusted to 2-3 with 2 N dilute hydrochloric acid, and the mixture was extracted three times with dichloromethane again. The organic phases were combined, dried over anhydrous sodium sulfate, concentrated to dryness at 40° C., and subjected to hot dissolution and cold crystallization in 1.6 L of an isopropanol-ethyl acetate (3:1) mixed solvent to give synthetic three-arm polyethylene glycol monopropionic acid monoamine with a molecular weight of 5000.

NMR (DMSO) 2.45 (t, CH₂COOCH₃)), 2.98 ppm (t, CH₂—NH—), 3.7 (t, CH₂CH₂COOCH₃),

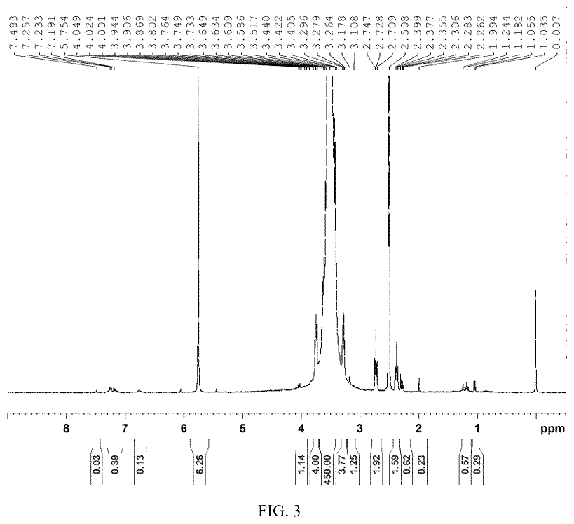

the nuclear magnetic resonance spectrum of intermediate 5 is shown in FIG. 3.

### 1.5: Synthesis of Three-Arm polyethylene glycol-methyl monopropionate-monoamine

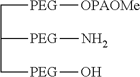

### Intermediate 6

Procedure: The starting material was three-arm polyethylene glycol monopropionic acid monoamine with a molecular weight of 5000. The synthesis procedure was the same as that described in step 1.2.

NMR (DMSO): 2.45 (t, CH₂COOCH₃), 2.98 ppm (t, CH₂—NH—), 3.32 (S, CH₂COOCH₃) 3.7 (t, CH₂CH₂COOCH₃)

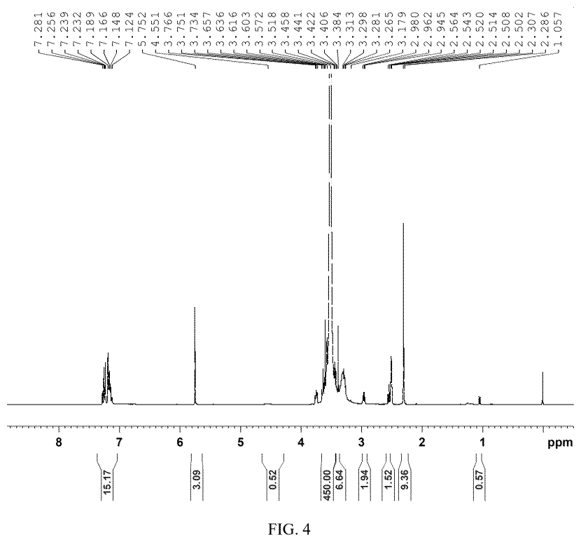

The nuclear magnetic resonance spectrum of intermediate 6 is shown in FIG. 4.

### 1.6: Synthesis of Three-Arm polyethylene glycol-methyl monopropionate-mono-tert-butoxycarbonylamine

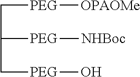

### Intermediate 7

Procedure: A three-necked round bottom flask was purged with nitrogen, and 70 g of three-arm polyethylene glycol methyl monopropionate monoamine, 3.9 g of Boc₂O, 2.0 g of triethylamine, and 350 mL of dichloromethane were added. The reaction mixture was stirred overnight. The system was concentrated the next day, subjected to hot dissolution and cold crystallization in 1.4 L of an isopropanol-ethyl acetate (3:1) mixed solvent, and dried under vacuum to give three-arm polyethylene glycol methyl monopropionate mono-tert-butoxycarbonylamine.

NMR (DMSO): 1.32 (S, —CH₃) 2.45 (t, CH₂COOCH₃), 3.32 (S, CH₂COOCH₃), 3.7 (t, CH₂CH₂COOCH₃)

### 1.7: Synthesis of Three-Arm polyethylene glycol-methyl monopropionate-mono-tert-butoxycarbonylamine-monomethanesulfonate

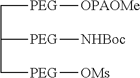

### Intermediate 8

Procedure: The starting material was three-arm polyethylene glycol methyl monopropionate mono-tert-butoxycarbonylamine with a molecular weight of 5000. The synthesis procedure was the same as that described in step 1.3.

NMR (DMSO): 1.32 (S, —CH₃), 2.45 (t, CH₂COOCH₃), 3.17 (S, CH₂OOSO₂CH₃), 3.7 (t, CH₂CH₂COOCH₃)

The nuclear magnetic resonance spectrum of intermediate 8 is shown in FIG. 5.

### 1.8: Synthesis of Three-Arm polyethylene glycol-methyl monopropionate-mono-tert-butoxycarbonylamine-monothiol-2-thiopyridine

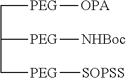

### Intermediate 11

Procedure: A three-necked round bottom flask was purged with nitrogen, and 60 g of three-arm polyethylene glycol methyl monopropionate mono-tert-butoxycarbonylamine monomethanesulfonate, 4.5 g of thiourea, and 600 mL of ethanol were added. The mixture was heated to reflux and allowed to react overnight. The reaction system was concentrated the next day. 450 mL of degassed water, 16 g of a sodium hydroxide solid, and 8 g of dithiothreitol were added. The mixture was allowed to react overnight under a nitrogen atmosphere. The pH of the system was adjusted to 5-6 with 2 N dilute hydrochloric acid the next day. The mixture was extracted three times with ethyl acetate to remove impurities. 60 g of sodium chloride was added, and the mixture was extracted three times with dichloromethane. The organic phases were combined, filtered through anhydrous sodium sulfate, and concentrated to give three-arm polyethylene glycol monopropionic acid mono-tert-butoxycarbonylamine monothiol. 40 g of 2,2′-dithiopyridine and 400 mL of MeOH were added to a three-necked flask and stirred until completely dissolved. The obtained three-arm polyethylene glycol monopropionic acid mono-tert-butoxycarbonylamine monothiol was dissolved in 0.7 L of methanol, and a solution of 2,2′-dithiopyridine in methanol was slowly added dropwise. The mixture was allowed to react overnight. The system was concentrated to dryness the next day. 0.7 L of degassed water was added to dissolve the residue. The mixture was extracted three times with ethyl acetate. 120 g of sodium chloride was added, and the mixture was extracted three times with dichloromethane. The organic phases were combined, filtered through anhydrous sodium sulfate, and concentrated to give a viscous oil. The residue was subjected to hot dissolution and cold crystallization in 1.4 L of an isopropanol-ethyl acetate (3:1) mixed solvent and dried under vacuum to give three-arm polyethylene glycol monopropionic acid mono-tert-butoxycarbonylamine monothiol-2-thiopyridine.

NMR (DMSO) 1.32 (S, —CH₃), 7.12, 7.68, 7.75, and 8.47 ppm (4m, pyridinyl protons, 4H).

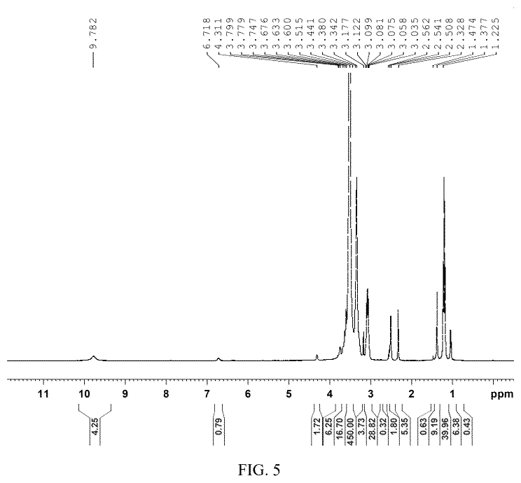

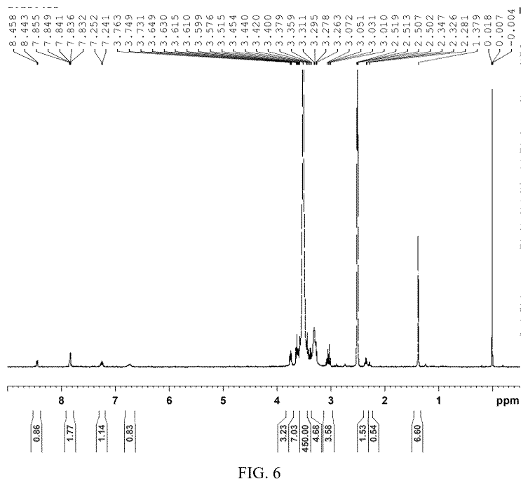

The nuclear magnetic resonance spectrum of intermediate 11 is shown in FIG. 6.

### 1.9: Synthesis of Three-Arm polyethylene glycol-monopropionic acid-NHS-mono-tert-butoxycarbonylamine-monothiol-2-thiopyridine

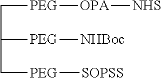

### Intermediate 12

Procedure: A three-necked round bottom flask was purged with nitrogen, and 30 g of three-arm polyethylene glycol monopropionic acid mono-tert-butoxycarbonylamine monothiol-2-thiopyridine, 1.0 g of hydroxysuccinimide, and 300 mL of dichloromethane were added and thoroughly dissolved. 1.5 g of dicyclohexylcarbodiimide was added in batches, and the mixture was allowed to react overnight. The next day, the mixture was filtered under vacuum through celite. The filtrate was concentrated by rotary evaporation to dryness. The residue was subjected to hot dissolution and cold crystallization in 600 mL of an isopropanol-ethyl acetate (3:1) mixed solvent and dried under vacuum to give three-arm polyethylene glycol monopropionic acid-NHS mono-tert-butoxycarbonylamine monothiol-2-thiopyridine.

NMR (DMSO) 1.32 (S, —CH₃), 2.83 (S, CH₂ on NHS), 7.12, 7.68, 7.75, and 8.47 ppm (4m, pyridinyl protons, 4H).

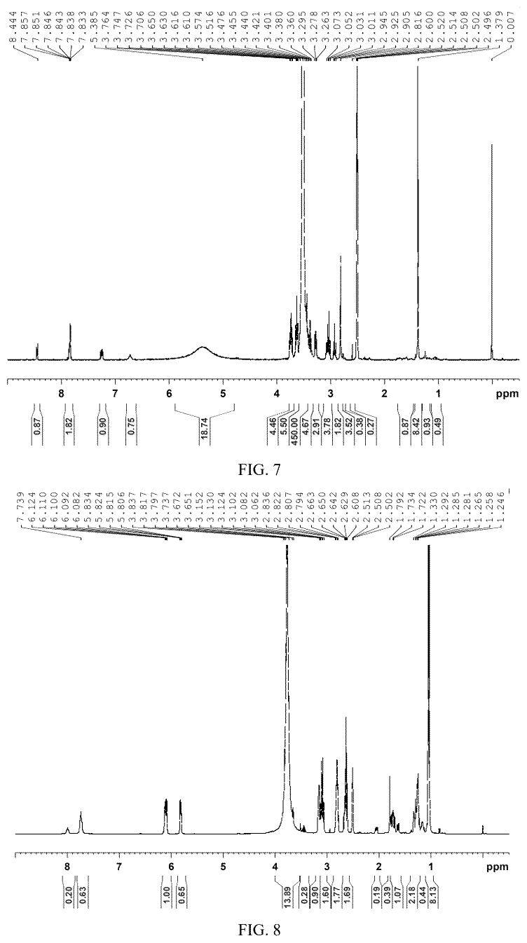

The nuclear magnetic resonance spectrum of intermediate 12 is shown in FIG. 7.

### 1.10: Synthesis of norbornene-2-formamidoethylamine

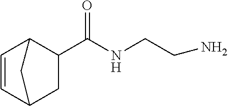 [SMILES: NCCNC(=O)C1CC2C=CC1C2]

Procedure: A three-necked round bottom flask was purged with nitrogen, and 5.1 g of norbornene-2-carboxylic acid, 4.72 g of NHS, and 9.16 g of DCC were added. The reaction was detected by TLC. After completion of the reaction, the mixture was filtered under vacuum to remove the insoluble substances. 4.8 g of Boc-amino-ethylamine and 3.6 g of triethylamine were added. After completion of the reaction as detected by TLC, the mixture was concentrated to dryness. Boc deprotection was performed with 3 M dilute hydrochloric acid, and the reaction was monitored by TLC until completion. The aqueous phase was extracted with a DCM:IPA (10:1) mixed solvent until no product remained. The organic phase was dried over anhydrous sodium sulfate and concentrated to dryness to give a viscous oil.

NMR (DMSO) 6.00, 6.20 (CH═CH)

The nuclear magnetic resonance spectrum of norbornene-2-formamidoethylamine is shown in FIG. 8.

### 1.11: Synthesis of Three-Arm polyethylene glycol-monopropionylaminoethylaminenorbornene-mono-tert-butoxycarbonylamine-monothiol-2-thiopyridine

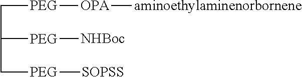

### Intermediate 13

Procedure: A three-necked round bottom flask was purged with nitrogen, and 8.0 g of intermediate 12 (three-arm polyethylene glycol monopropionic acid-NHS mono-tert-butoxycarbonylamine monothiol-2-thiopyridine), 0.31 g of norbornene-2-formamidoethylamine, and 0.24 g of triethylamine were added. The reaction mixture was stirred overnight. The system was concentrated to dryness the next day. The concentrated residue was dissolved in 200 mL of water. The mixture was extracted three times with 50 mL of ethyl acetate and three times with 50 mL of dichloromethane, filtered through anhydrous sodium sulfate, and concentrated to give a viscous oil. The residue was subjected to hot dissolution and cold crystallization in 160 mL of an isopropanol-ethyl acetate (3:1) mixed solvent and dried under vacuum to give three-arm polyethylene glycol monopropionylaminoethylaminenorbornene mono-tert-butoxycarbonylamine monothiol-2-thiopyridine.

NMR (DMSO), 1.37 (S, —CH₃), 6.00, 6.20 (m, CH═CH), 7.12, 7.68, 7.75, and 8.47 ppm (m, pyridinyl protons, 4H).

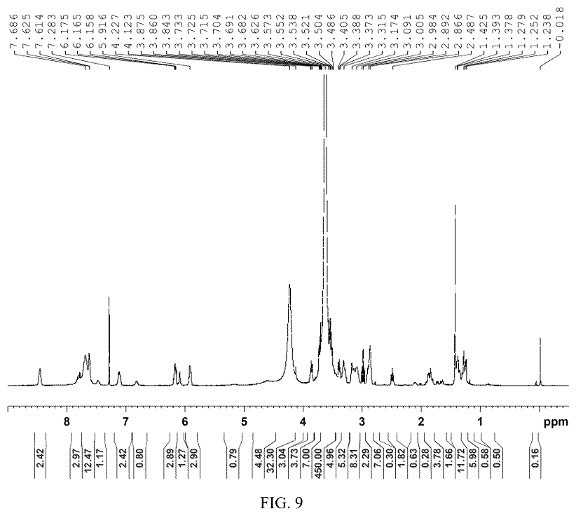

The nuclear magnetic resonance spectrum of intermediate 13 is shown in FIG. 9.

### 1.12: Synthesis of Three-Arm polyethylene glycol-monopropionylaminoethylaminenorbornene-monoamino-monothiol-2-thiopyridine

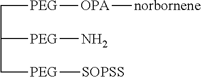

### Intermediate 14

Procedure: A three-necked round bottom flask was purged with nitrogen, and 5.0 g of intermediate 13 (three-arm polyethylene glycol monopropionylaminoethylaminenorbornene mono-tert-butoxycarbonylamine monothiol-2-thiopyridine) and 80 mL of 3 N dilute hydrochloric acid were added. After 1 h, the mixture was extracted three times with 20 mL of ethyl acetate and three times with 20 mL of dichloromethane, filtered through anhydrous sodium sulfate, and concentrated to give a viscous oil. The residue was subjected to hot dissolution and cold crystallization in 100 mL of an isopropanol-ethyl acetate (1:3) mixed solvent and dried under vacuum to give three-arm polyethylene glycol monopropionylaminoethylaminenorbornene monoamino monothiol-2-thiopyridine.

NMR (DMSO) 6.00, 6.20 (m, CH═CH), 7.12, 7.68, 7.75, and 8.47 ppm (m, pyridinyl protons, 4H).

The nuclear magnetic resonance spectrum of intermediate 14 is shown in FIG. 10.

### 1.13: Synthesis of Three-Arm polyethylene glycol-monopropionylaminoethylaminenorbornene-monoamino DBCO-monothiol-2-thiopyridine

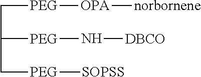

Procedure: A three-necked round bottom flask was purged with nitrogen, and 3.0 g of intermediate 14 (three-arm polyethylene glycol-monopropionylaminoethylaminenorbornene-monoamino-monothiol-2-thiopyridine), 360 mg of DBCO-NHS, 122 mg of triethylamine, and 30 mL of dichloromethane were added. The reaction mixture was stirred overnight. The system was concentrated to dryness the next day. The concentrated residue was dissolved in 90 mL of water. The mixture was extracted three times with 50 mL of ethyl acetate and three times with 50 mL of dichloromethane, filtered through anhydrous sodium sulfate, and concentrated to give a viscous oil. The residue was subjected to hot dissolution and cold crystallization in 60 mL of an isopropanol-ethyl acetate (1:3) mixed solvent and dried under vacuum to give three-arm polyethylene glycol monopropionylaminoethylaminenorbornene monoamino DBCO monothiol-2-thiopyridine.

NMR (DMSO) 6.00, 6.20 (m, CH═CH), 7.0-8.0 ppm (m, pyridinyl protons, 4H; DBCO, 8H).

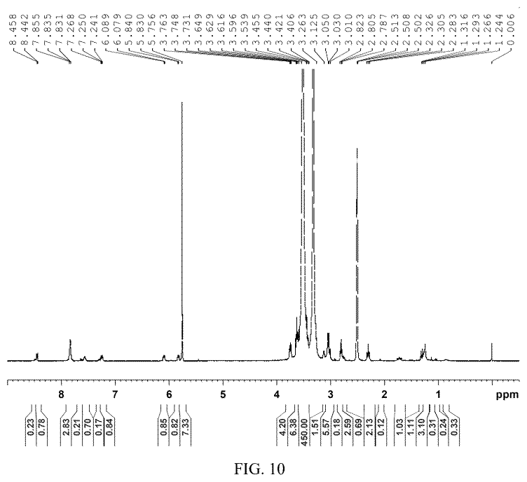

The nuclear magnetic resonance spectrum of three-arm polyethylene glycol-monopropionylaminoethylaminenorbornene-monoamino DBCO-monothiol-2-thiopyridine is shown in FIG. 11.

### 1.14: Synthesis of Three-Arm polyethylene glycol-monopropionylaminoethylaminenorbornene-monoamino DBCO-monothiol (3ARM-NB-DBCO-SH)

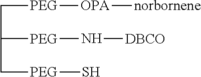

Procedure: A three-necked round bottom flask was purged with nitrogen, and 3.0 g of intermediate 14 (three-arm polyethylene glycol-monopropionylaminoethylaminenorbornene-monoamino DBCO-monothiol-2-thiopyridine) was added and dissolved in 30 mL of PBS (pH 7.4). The mixture was stirred, and 277 mg of DTT was added. The mixture was allowed to react at room temperature overnight. The pH of the system was adjusted to 4-5 with 1 N HCl. 4.5 g of sodium chloride was added. The mixture was extracted three times with 15 mL+12 mL+10 mL of dichloromethane, dried over anhydrous sodium sulfate, and filtered.

The filtrate was concentrated at 40° C. using a rotary evaporator until no liquid droplets were observed. 60 mL of isopropanol was added, and the mixture was precipitated in an ice bath and filtered. The filter cake was washed twice with isopropanol and dried under vacuum to give three-arm polyethylene glycol monopropionylaminoethylaminenorbornene monoamino DBCO monothiol.

NMR (DMSO) 6.00, 6.20 (m, CH═CH), 7.0-8.0 ppm (DBCO, 8H), 1.0-2.0 ppm (SH, 1H).

### 1.15: Preparation of Anti-CD3 Nanobody-PEG3500-N3 PEG Monosubstituted Antibody

### (1) Preparation of PEGylation Reaction Mixture

An appropriate amount of anti-CD3 nanobody was taken and subjected to buffer exchange into a 5 mM PB buffer (pH 7.0) using a G25 Desalting column. The concentration was determined using a BCA protein quantification kit. A reaction mixture was prepared from anti-CD3 nanobody and N3-PEG3500-SPA at a reaction molar ratio of 1:4 (i.e., anti-CD3 nanobody:N3-PEG3500-SPA). The mixture was mixed well, placed on a shaking bed at 25° C., and allowed to react for 2 h.

### (2) Separation of PEG Monosubstituted CD3 Antibody Using Q Sepharose High Performance Resin

First, the reaction mixture obtained in step (1) was subjected to buffer exchange into a 10 mM Tris-HCl buffer (pH 10.0) using a G25 Desalting column.

Second, the resin was equilibrated with mobile phase A (10 mM Tris-HCl buffer, pH 10.0). The sample was loaded at a flow rate of 2 mL/min, followed by equilibration with phase A. Step gradient elution was performed using mobile phase B (10 mM Tris-HCl+1 M NaCl buffer, pH 10.0). Elution fractions corresponding to 20%-30% of the phase B concentration were collected.

Third, the collected elution fractions were subjected to SDS-PAGE and HPLC purity analysis. Fractions with purity higher than 99.5% were combined and stored in a PBS buffer.

### 1.16: Preparation of Anti-EGFR Nanobody-PEG3500-MAL PEG Monosubstituted Antibody

### (1) Preparation of PEGylation Reaction Mixture

An appropriate amount of anti-EGFR nanobody was taken and subjected to buffer exchange into a 5 mM PB buffer (pH 7.0) using a G25 Desalting column. The concentration was determined using a BCA protein quantification kit. A reaction mixture was prepared from anti-EGFR nanobody and MAL-PEG3500-SPA at a reaction molar ratio of 1:5 (i.e., anti-EGFR nanobody:MAL-PEG3500-SPA). The mixture was mixed well, placed on a shaking bed at 25° C., and allowed to react for 2 h.

### (2) Separation of PEG Monosubstituted EGFR Antibody Using Q Sepharose High Performance Resin

First, the reaction mixture obtained in step (1) was subjected to buffer exchange into a 10 mM Tris-HCl buffer (pH 10.0) using a G25 Desalting column.

Second, the resin was equilibrated with mobile phase A (10 mM Tris-HCl buffer, pH 10.0). The sample was loaded at a flow rate of 2 mL/min, followed by equilibration with phase A. Step gradient elution was performed using mobile phase B (10 mM Tris-HCl+1 M NaCl buffer, pH 10.0). Elution fractions corresponding to 5%-10% of the phase B concentration were collected.

Third, the collected elution fractions were subjected to SDS-PAGE and HPLC purity analysis. Fractions with purity higher than 99.5% were combined and stored in a PBS buffer.

### 1.17: Preparation of Anti-CD28 Nanobody-PEG3500-MTZ PEG Monosubstituted Antibody

### (1) Preparation of PEGylation Reaction Mixture

An appropriate amount of anti-CD28 nanobody was taken and subjected to buffer exchange into a 5 mM PB buffer (pH 7.0) using a G25 Desalting column. The concentration was determined using a BCA protein quantification kit. A reaction mixture was prepared from anti-CD28 nanobody and MTZ-PEG3500-SPA at a reaction molar ratio of 1:5 (i.e., anti-CD28 nanobody:MTZ-PEG3500-SPA). The mixture was mixed well, placed on a shaking bed at 25° C., and allowed to react for 2 h.

### (2) Separation of PEG Monosubstituted EGFR Antibody Using CM Sepharose High Performance Resin

First, the reaction mixture obtained in step (1) was subjected to buffer exchange into a 10 mM NaAC-HAC buffer (pH 6.0) using a G25 Desalting column.

Second, the resin was equilibrated with mobile phase A (10 mM NaAC-HAC buffer, pH 6.0). The sample was loaded at a flow rate of 2 mL/min, followed by equilibration with phase A. Step gradient elution was performed using mobile phase B (10 mM NaAC-HAC+1 M NaCl buffer, pH 6.0). Elution fractions corresponding to 7%-10% of the phase B concentration were collected.

Third, the collected elution fractions were subjected to SDS-PAGE and HPLC purity analysis. Fractions with purity higher than 99.5% were combined and stored in a PBS buffer.

### 2. Conjugation of PEGylation Antibodies with Different Functional Groups to 3ARM-DBCO-SH-Norbornene PEG and Separation of Trispecific Antibody Compound

### (1) Preparation of Reaction Mixture

First, 3ARM-DBCO-SH-Norbornene PEG was dissolved in PBS.

Second, anti-EGFR nanobody-PEG3500-MAL was added to the reaction system, and the mixture was allowed to react at room temperature for 2 h to complete the conjugation of MAL to SH.

Third, anti-CD3 nanobody-PEG3500-N3 was added to the reaction system, and the mixture was allowed to react at room temperature for 2 h to complete the conjugation of N3 to DBCO.

Finally, anti-CD28 nanobody-PEG3500-MTZ was added to the reaction system, and the mixture was allowed to react at room temperature for 2 h to complete the conjugation of TCO to Tetrazine.

### (2) Separation of Trispecific Antibody Compound

First, the reaction mixture obtained in step (1) was subjected to buffer exchange into a 10 mM Tris-HCl buffer (pH 10.0) using a G25 Desalting column.

Second, the Q Sepharose High Performance resin was equilibrated with mobile phase A (10 mM Tris-HCl buffer, pH 10.0). The sample was loaded at a flow rate of 2 mL/min, followed by equilibration with phase A. Linear gradient elution was performed using mobile phase B (10 mM Tris-HCl+1 M NaCl buffer, pH 10.0). Elution fractions corresponding to 5%-10% of the phase B concentration were collected.

Third, the collected elution fractions were subjected to SDS-PAGE and HPLC purity analysis. Fractions with purity higher than 99.9% were combined and stored in a PBS buffer.

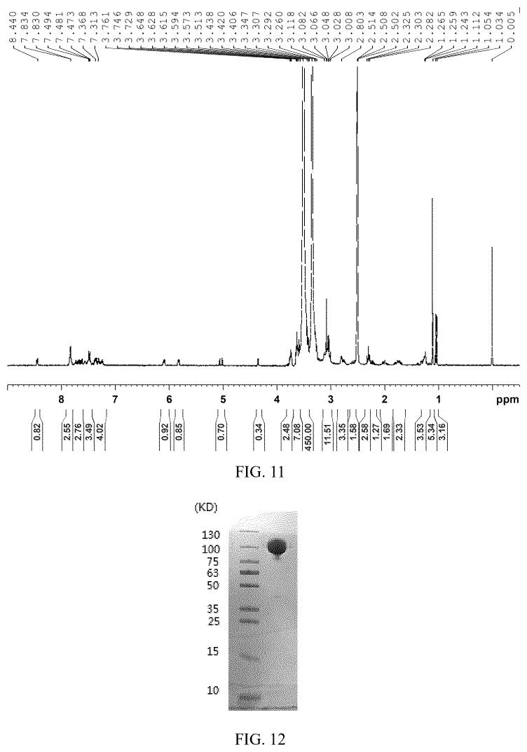

### 3. Purity Analysis Results for Trispecific Antibody Formed Via PEG Conjugation (See FIG. 12)

### 4. Binding Ability of CD3-CD28-EGFR Trispecific Antibody to Target Cells

(1) A431 cells in good growth condition were collected and prepared into a single-cell suspension. The cell density was adjusted to 1×10⁶ cell/mL, and the suspension was added to 1.5 mL centrifuge tubes at 1 mL/tube and centrifuged at 1000 rpm for 3 min. The supernatant was discarded.
(2) The cells were resuspended in 100 μL of complete medium. The FITC-labeled EGFR monoclonal antibody and the FITC-labeled trispecific monoclonal antibody were added according to the experimental design, while no antibody was added to the blank control check. The mixture was thoroughly mixed using a pipettor and incubated in a refrigerator at 4° C. for 30 min.
(3) The centrifuge tubes were removed from the refrigerator and centrifuged at 1000 rpm for 3 min. The cells were washed with 1 mL of PBS, and this process was repeated 3 times. The cells were resuspended in 300 μL of PBS and analyzed by flow cytometry.

### Experimental Results:

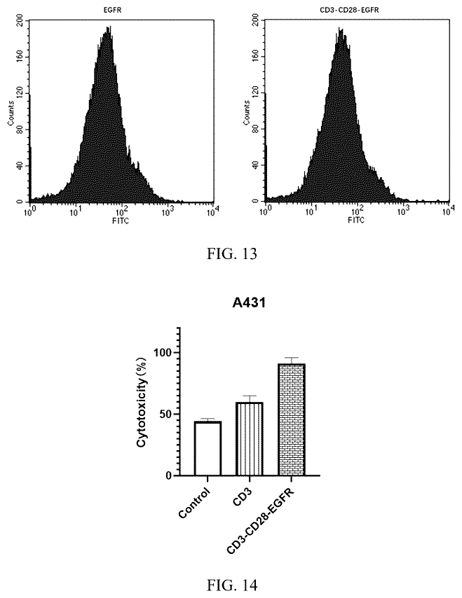

Flow cytometry results revealed that the A431 cell surface highly expressed EGFR protein. Subsequently, the binding abilities of the trispecific antibody and the EGFR monoclonal antibody to target cells were compared. As shown in FIG. 13, both the trispecific antibody and the EGFR monoclonal antibody exhibited a relatively strong binding ability to A431 cells.

### 5. Effect of CD3-CD28-EGFR Trispecific Antibody on T Cell Killing Activity

### 5.1 Pan-T Cell Isolation from PBMCs

(1) One vial of PBMCs was thawed in 10 mL of complete medium. 100 μL of the cell suspension was taken for cell counting. The required cell quantity was calculated based on cell density. The remaining suspension was centrifuged at 300 g for 8 min.
(2) After centrifugation, PBMCs were labeled with antibodies according to the instructions of the Miltenyi Pan-T cell negative selection kit.
(3) The labeled cell suspension was loaded into the sample column of the Miltenyi magnetic sorting apparatus. The negative selection program was set for sorting. The cells obtained from the negative selection column were Pan-T cells.
(4) PE-labeled anti-human CD3 antibody and its isotype control antibody were used to stain Pan-T cells and PBMCs, respectively. The sorting results were analyzed by flow cytometry.
(5) Pan-T cells were cryopreserved at 5×10⁷ cells/mL, with 1 mL per tube.

### 5.2 Detection of Trispecific Antibody-Mediated T Cell Killing Ability Against Tumor Cells by CCK8 Assay

(1) A431 cells in good growth condition were taken and added to a 96-well plate at 1×10⁴ cells/100 μL. The 96-well plate was placed in a cell incubator and cultured for 18-24 h.
(2) After complete cell adherence, the original medium in the 96-well plate was discarded. 5×10⁴ Pan-T cells and the antibodies according to the experimental design were added, with the final antibody concentration set to 100 ng/mL. The final volume per well of the 96-well plate was 200 μL. The 96-well plate was placed back into the cell incubator and further cultured for 48 h.
(3) After 48 h, the 96-well plate was taken out. 120 μL of liquid from each well was aspirated using a multichannel pipettor and transferred to a new 96-well plate, which was stored in an ultra-low temperature refrigerator at −80° C. for later use. The remaining liquid was aspirated, and each well was washed twice with PBS pre-warmed to 37° C. Subsequently, 100 μL of complete medium containing a 10% CCK8 solution was added to each well. The 96-well plate was placed back into the incubator and incubated for 4 h.
(4) After 4 h, the 96-well plate was taken out and placed into a microplate reader. The program was set to measure the absorbance of each well at 450 nm. The results were visualized using the GraphPad Prism software.

### Experimental Results:

To detect the killing activity (cytotoxicity) of trispecific antibody-mediated T cells against target cells, the human skin cancer cell line A431 with a relatively high EGFR expression level was selected as target cells, and isolated Pan-T cells were used as effector cells. The experiment was conducted with an effector-to-target cell ratio of 5:1 and an antibody working concentration of 100 ng/mL. As shown in FIG. 14, the trispecific antibody-mediated T cells exhibited a target cell killing rate of 95%, while T cells without any antibody addition exhibited a killing rate of about 45%. The trispecific antibody significantly enhanced T cell killing ability against target cells, and the difference was statistically significant compared with the control check. 6. Effect of Trispecific Antibody on IL-2 Secretion by T Cells
(1) The microplate was coated overnight with IL-2 capture antibody diluted in a coating buffer. The plate was placed in a refrigerator at 4° C. overnight.
(2) The cell supernatants collected from the previous experiment were taken out from the −80° C. refrigerator and thawed in a refrigerator at 4° C.
(3) The overnight-coated microplate was taken out from the refrigerator. The liquid in the wells was discarded, and the wells were washed 3 times with a washing buffer. 200 μL of a blocking buffer was added, and the plate was blocked for 1 h.
(4) After 1 h, the liquid in the wells was discarded. The wells were washed 3 times with a washing buffer. 100 μL of IL-2 standards or samples at different concentrations was added to each well, followed by incubation at room temperature for 2 h.
(5) After 2 h, the liquid in the wells was discarded. The wells were washed 5 times with a washing buffer. Subsequently, 100 μL of the detection antibody and the horseradish peroxidase-labeled secondary antibody was added, followed by incubation at room temperature for 1 h.
(6) After 1 h, the liquid in the wells was discarded. The wells were washed 7 times with a washing buffer (1 min interval between each wash). Subsequently, 100 μL of a substrate solution was added to each well, followed by incubation at room temperature in the dark for 15-30 min.
(7) When color developed in the wells, 50 μL of a stopping solution was added to each well. The plate was placed in a microplate reader and shaken for 30 s, and the absorbance at 450 nm was measured. Results were visualized using the GraphPad Prism software.

### Experimental Results:

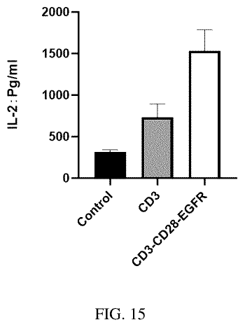

To detect cytokine IL-2 secretion during the killing process, a portion of the supernatant was pipetted, and IL-2 secreted by Pan-T cells into the supernatant was measured via ELISA. As shown in FIG. 15, the IL-2 secretion level was about 1500 μg/mL when the trispecific antibody was used, whereas the IL-2 secretion level was about 320 μg/mL without antibody treatment. This indicated that the IL-2 secretion level when the trispecific antibody was used was significantly higher than that without any antibody treatment, and the difference was statistically significant. These results suggest that the trispecific antibody can promote IL-2 secretion by T cells, thereby more effectively activating relevant immune cells in the body and promoting the attack and killing against target cells.

1-21. (canceled)

22. A multi-arm polyethylene glycol-drug conjugate, wherein the multi-arm polyethylene glycol-drug conjugate has a structure represented by general formula I: 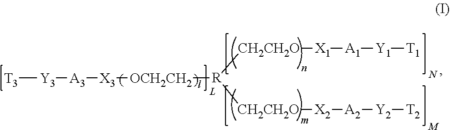
wherein R is a central molecule selected from one of a polyol residue, an oligopeptide residue, an amino acid residue, and an amino acid residue derivative;
n, m, and 1 are each independently selected from an integer of 0-228;
X₁, X₂, and X₃ are all linking groups;
A₁, A₂, and A₃ are identical or different polyethylene glycol residues, separately;
Y₁, Y₂, and Y₃ are all linking groups;
T₁, T₂, and T₃ are each independently selected from an antibody, an antibody fragment, and a derivative thereof;
N, M, and L are each independently selected from an integer of 1-24, and N+M+L≥3; and
T₁, T₂, and T₃ are different from each other.

23. The multi-arm polyethylene glycol-drug conjugate according to claim 22, wherein the polyol residue is selected from one of glycerol, polyglycerol, pentaerythritol, polypentaerythritol, a mannitol residue or a glycerol ether group thereof, methylglucoside, and sucrose.

24. The multi-arm polyethylene glycol-drug conjugate according to claim 22, wherein the amino acid residue has amino, carboxyl, and sulfydryl simultaneously.

25. The multi-arm polyethylene glycol-drug conjugate according to claim 22, wherein n, m, and 1 are each independently selected from an integer of 0-80.

26. The multi-arm polyethylene glycol-drug conjugate according to claim 22, wherein X₁, X₂, and X₃ are each independently selected from the group consisting of —(CH₂)ₐ—, —(CH₂)_{b}NH, —CO(CH₂)_{c}CO—, —(CH₂)_{d}CO—, —NH(CH₂)ₑNHCO—, 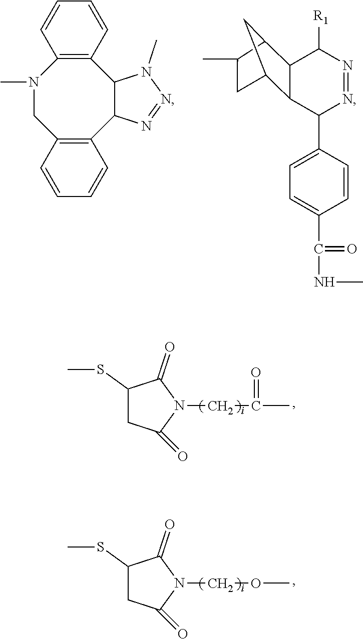
—NH—, —(CH₂)_{f}OCONH—, —CONH(CH₂)_{g}, —(CH₂)ₕNHCO—, —(CH₂)ⱼCONH—, and a combination thereof, wherein a, b, c, d, e, f, g, h, i, and j are each independently selected from an integer of 0-10, and R₁ is C₁₋₁₀ linear/branched alkyl; optionally, C atoms on X₁, X₂, and X₃ are substituted with alkyl, alkoxy, amino, hydroxy, aryl, and heteroaryl.

27. The multi-arm polyethylene glycol-drug conjugate according to claim 26, wherein a is an integer selected from 0-5; b is an integer selected from 0-5; c is an integer selected from 0-5; i is an integer selected from 0-5; j is an integer selected from 0-5; g is an integer selected from 0-5; and h is an integer selected from 0-5.

28. The multi-arm polyethylene glycol-drug conjugate according to claim 22, wherein X₂ is selected from

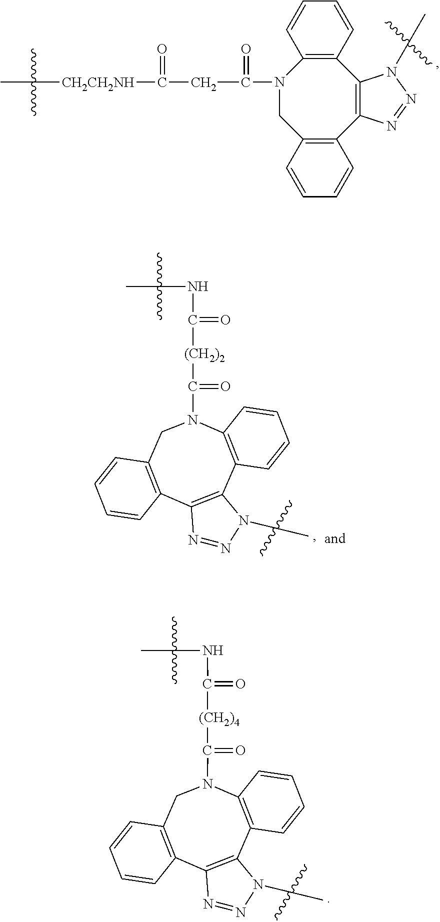

29. The multi-arm polyethylene glycol-drug conjugate according to claim 22, wherein X₃ is selected from:

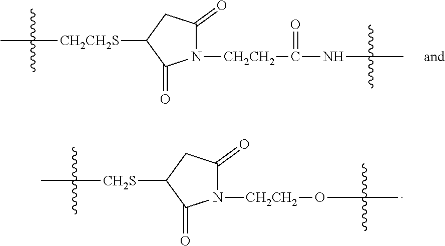

30. The multi-arm polyethylene glycol-drug conjugate according to claim 22, X₁ is

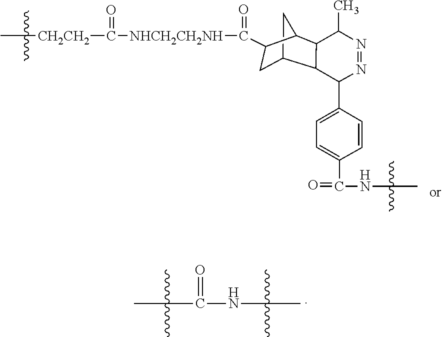

31. The multi-arm polyethylene glycol-drug conjugate according to claim 22, wherein A₁, A₂, and A₃ comprise a —(CH₂CH₂O)ₚ— repeating unit, wherein P is an integer selected from 0-250; A₁, A₂, and A₃ have a molecular weight of 0-11000 Da, separately; and A₁, A₂, and A₃ are each independently selected from linear, Y-shaped, and multi-branched polyethylene glycol residues.

32. The multi-arm polyethylene glycol-drug conjugate according to claim 31, wherein P is an integer selected from 0-114.

33. The multi-arm polyethylene glycol-drug conjugate according to claim 22, wherein A₁, A₂, and A₃ have a molecular weight of 0-5000 Da, separately.

34. The multi-arm polyethylene glycol-drug conjugate according to claim 22, wherein Y₁, Y₂, and Y₃ are each independently selected from the group consisting of —(CH₂)ₖ₁—, —NH—, —S—S—, —(CH₂)ₖ₂CO—, —(CH₂)ₖ₃ NHCO—, 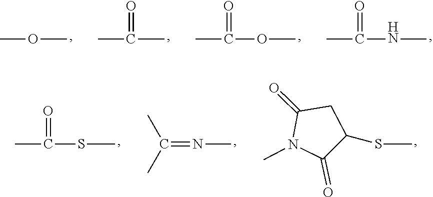
and a combination thereof, wherein K1, K2, and K3 are each independently selected from an integer of 1-10.

35. The multi-arm polyethylene glycol-drug conjugate according to claim 22, wherein Y₁, Y₂, and Y₃ are all —(CH₂)₂CO—NH—, or Y₁, Y₂, and Y₃ are all —(CH₂)CO—NH—.

36. The multi-arm polyethylene glycol-drug conjugate according to claim 22, wherein T₁, T₂, and T₃ are each independently selected from an anti-CD3 antibody, an anti-EGFR antibody, an anti-CD28 antibody, an anti-HER2 antibody, an anti-PMSA antibody, an anti-VEGFR antibody, an anti-CD30 antibody, an anti-CD22 antibody, an anti-CD56 antibody, an anti-CD29 antibody, an anti-GPNMB antibody, an anti-CD138 antibody, an anti-CD74 antibody, an anti-ENPP3 antibody, an anti-Nectin-4 antibody, an anti-EGFRVIII antibody, an anti-SLC44A4 antibody, an anti-mesothelin antibody, an anti-ET8R antibody, an anti-CD37 antibody, an anti-CEACAM5 antibody, an anti-CD70 antibody, an anti-MUC16 antibody, an anti-CD79b antibody, an anti-MUC16 antibody, and an anti-Muc1 antibody.

37. The multi-arm polyethylene glycol-drug conjugate according to claim 22, wherein the multi-arm polyethylene glycol-drug conjugate has a structure represented by general formula II or III:

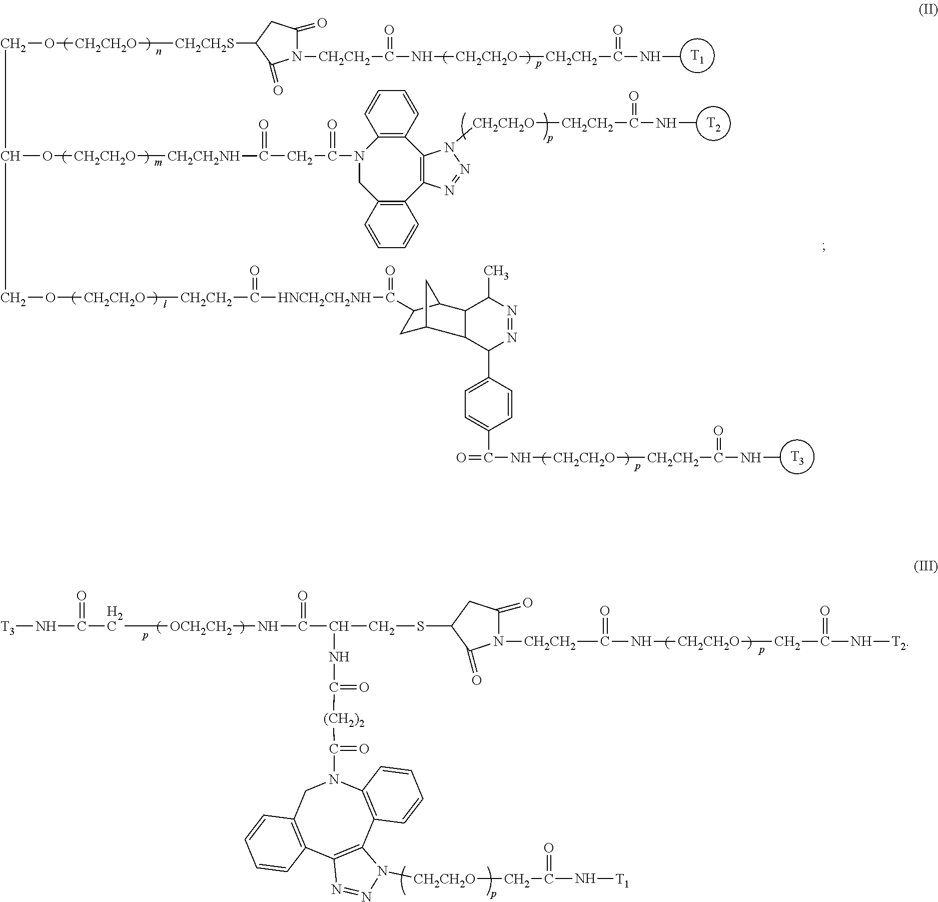

38. The multi-arm polyethylene glycol-drug conjugate according to claim 22, the multi-arm polyethylene glycol-drug conjugate has a structure represented by general formula IV or V:

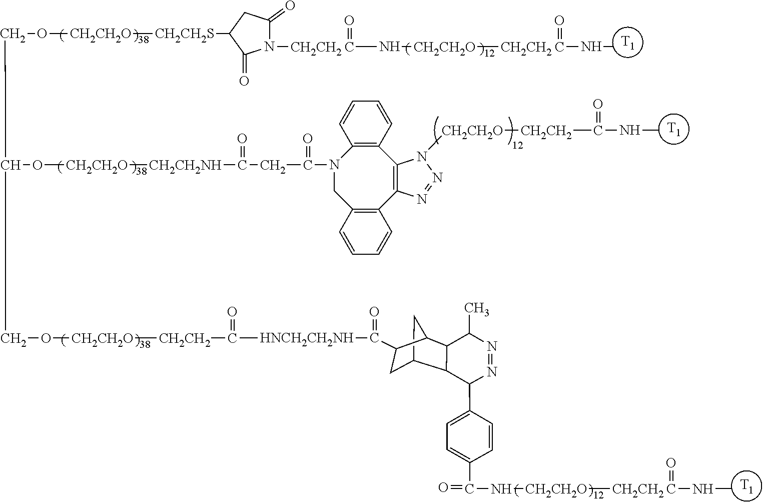

39. The multi-arm polyethylene glycol-drug conjugate according to claim 37, wherein the multi-arm polyethylene glycol-drug conjugate has a structure represented by formula VI, VII, VIII, or IX:

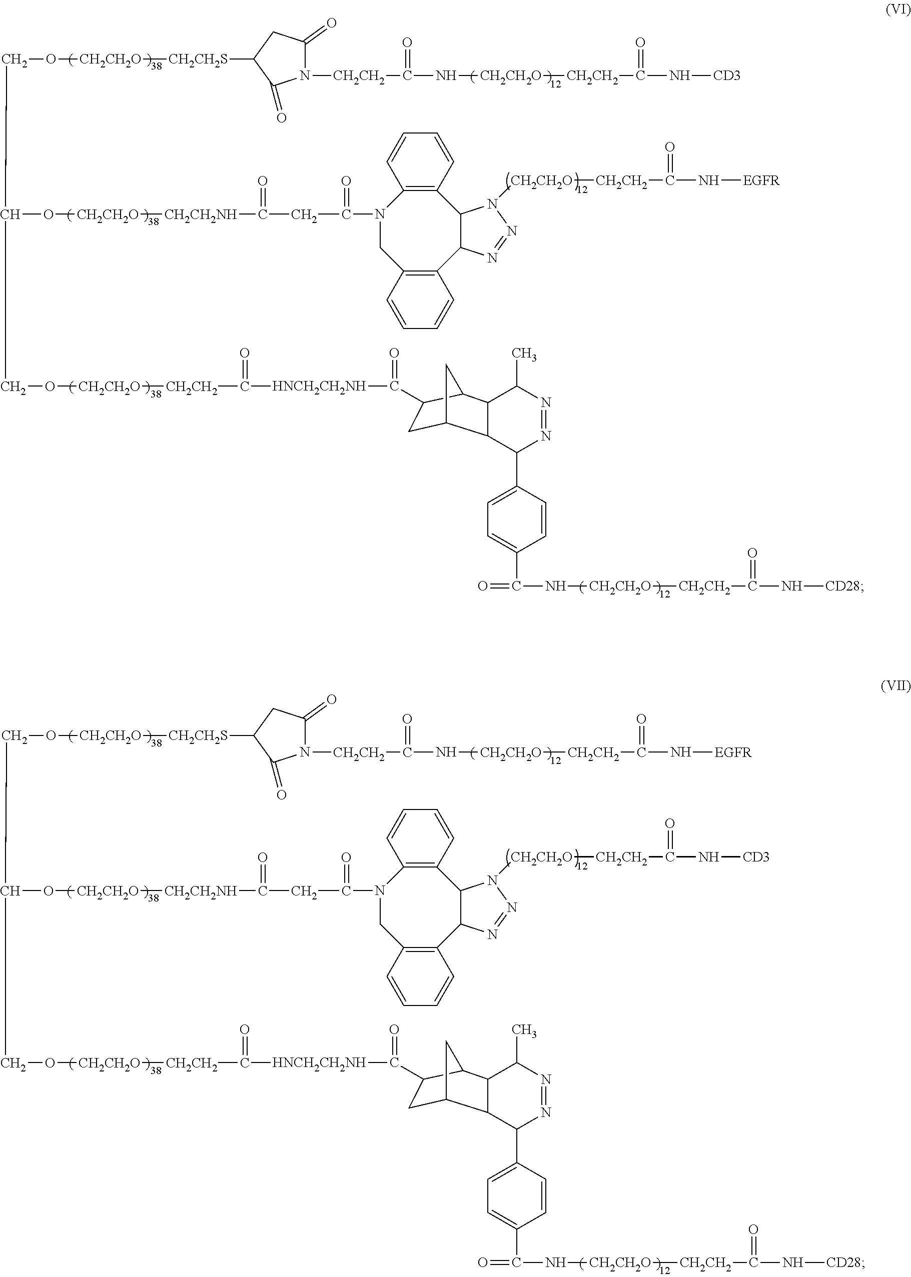

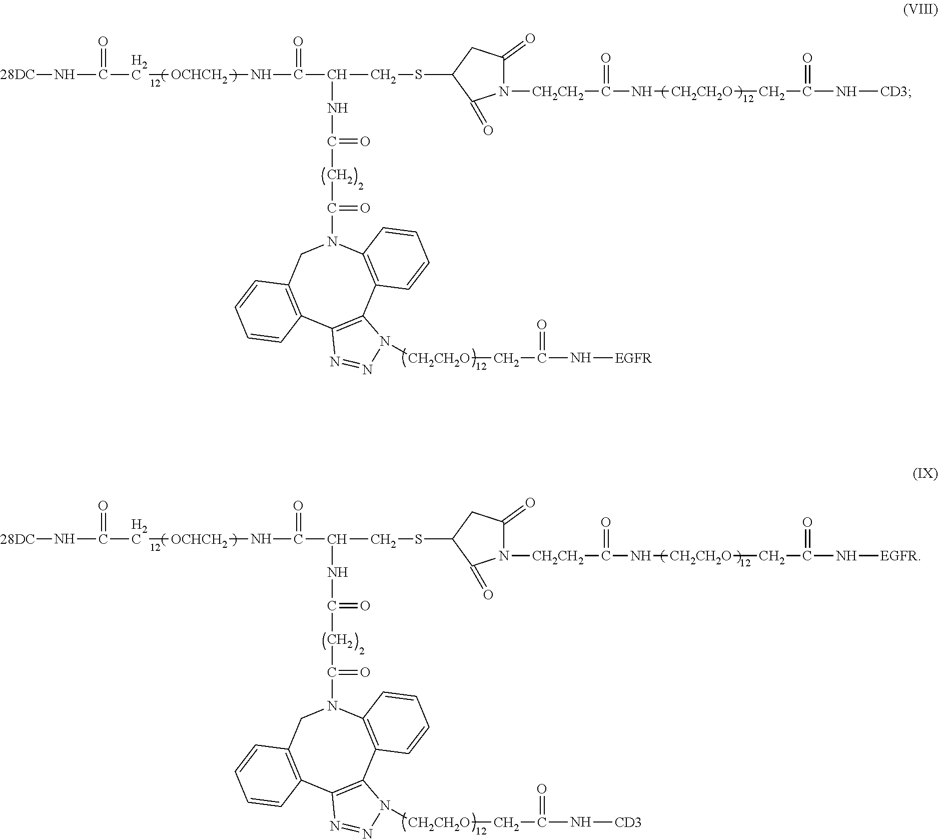

40. A pharmaceutical composition, comprising the multi-arm polyethylene glycol-drug conjugate according to claim 22.

41. Use of the multi-arm polyethylene glycol-drug conjugate according to claim 22 in targeted prevention and/or treatment of a disease.
